(19) United States
(12) Patent Application Publication
(10) Pub. No.: US 2006/0194229 A1
(43) Pub. Date: Aug. 31, 2006

(54) Cancer markers and detection methods

(75) Inventor: Don Adams North, Arlington, TX (US)

Correspondence Address: BAKER BOTTS L.L.P.;PATENT DEPARTMENT, 98 SAN JACINTO BLVD., SUITE 1500, AUSTIN, TX, 78701-4039, US

(73) Assignee: Sky Genetics, Inc.

(21) Appl. No.: 11/339,052

(22) Filed: Jan. 24, 2006

Related U.S. Application Data
(60) Provisional application No. 60/646,961, filed on Jan. 25, 2005.
(60) Provisional application No. 60/669,639, filed on Apr. 8, 2005.

(51) Int. Cl. C12Q 1/68; G01N 33/574

(52) U.S. Cl. 435/6; 435/7.23

(57) ABSTRACT

The present invention relates to cancer markers and methods of detecting cancer markers in a sample. The sample may be peripheral blood. Cancer markers are most commonly mutated or abnormal DNA sequences associated with metastatic cancer. Markers may be detected using PCR, microarrays, or other nucleic acid or peptide-based assays. These methods may be used for a variety of diagnostic purposes, including initial, early-stage or later diagnosis of cancer, particularly metastatic cancer and monitoring of cancer or treatment progression. The cancer markers may also be used to create a cancer marker profile. Treatment may be directed based on this profile. Additionally, methods using blood may provide a cancer marker profile of mutations or abnormalities found in at least one of several tumors in the body, instead of merely one tumor. The invention also include kits, such as primer kits, and microarrays for use in performing the various methods.

### PRIORITY CLAIM

The present application claims priority under 35 U.S.C. §119(e) to U.S. Provisional Patent Application Ser. No. 60/646961, filed Jan. 25, 2005, titled “Cancer Detection Reagents and Uses in Pathology and Diagnostics and Targeted Cancer Cell Death.” The present application also claims priority under 35 U.S.C. §119(e) to U.S. Provisional Patent Application Ser. No. 60/669639, filed Apr. 8, 2005, titled “Cancer Markers and Detection Methods.” The present application also claims priority under 35 U.S.C. §120 to U.S. application Ser. No. 11/311,594, filed Dec. 19, 2005, titled “Nucleic Acids for Apoptosis of Cancer Cells.” All three priority applications are incorporatef by reference herein.

### FIELD OF THE INVENTION

The present invention relates to methods of detecting cancer markers in the blood of a subject, such as a human suspected of having cancer. The invention more particularly relates to methods of detecting metastatic cancer or other cancers that release markers into the blood. It may be used for initial diagnosis and prognosis, treatment direction, and treatment or disease monitoring. Detection may be accomplished using cancer detection reagents corresponding to the cancer markers.

### BACKGROUND

Cancer results when a cell in the body malfunctions and begins to grow uncontrollably. These malfunctions result from mutations in the cell's DNA blueprint. Thus, while early cancer diagnosis focused on the growth properties and the physical appearance of suspected cancer cells, more modern techniques have begun to examine the cell's inner workings.

Not all cancers are caused by the same mutation. Some treatments that work well for particular cancer-causing mutations are ineffective against cancer having other types of mutations and may actually cause more harm than good if inappropriately prescribed. Thus, it is imperative that cancer diagnostics' ability to distinguish different types of cancer keep pace with the ability to treat different types of cancers appropriately. Current diagnostic methods are struggling to match the speed at which new treatments are developed.

Another problem with current cancer diagnostic methods lies in the need for tissue samples to analyze. All presently successful cancer diagnostic methods, other than pure imaging, require cancer cells to be removed from the patient's body. These cells are most commonly obtained from a tissue biopsy. While effective, tissue biopsies are expensive, time-consuming, and painful for the patient. Additionally, the time required to schedule and obtain a tissue biopsy then analyze it causes a delay in treatment and the biopsy process itself may release cancer cells into the blood stream, resulting in increased metastasis.

Even worse, in some cases a tissue biopsy is not possible due to the location of a tumor. In those instances, the exact nature of the cancer cannot be determined until after surgery has been performed and the tumor removed. While these post-operative tests are still useful in directing further treatment of the patient, if the nature of the tumor could be determined in advance, it might be much more feasible to try non-invasive treatments, such as chemotherapy, before putting a patient through the rigors of surgery. Even if surgery were required, the patient might still benefit from a more detailed pre-operative diagnosis. Such a diagnosis might, for example, allow pre-operative treatment with drugs designed to minimize the chance of metastatic spread of cancer cells dislodged from the tumor during surgery. It might also provide greater direction for surgical techniques, such as how much tissue surrounding the tumor to remove.

Currently, some of the most successful cell-based diagnostic methods utilize non-biopsy samples. For example, PAP smears look for cellular irregularities, but utilize cells normally sloughed off by the body. PAP smears continue to save thousands of lives each year by allowing easy and very early detection of cells in the process of becoming cervical cancer.

Because of problems associated with biopsies and the success of simpler methods, such as PAP smears, the medical community has spent years searching for cancer diagnostics using another readily available sample, blood, particularly peripheral blood. Their efforts have met with some success. For example, the progress or recurrence of prostate cancer is readily monitored using a blood test. However, current blood-based cancer diagnostics, like the prostate cancer test, still remain focused on particular types of cancer. The need remains for a cancer diagnostic able to use blood to diagnose a wide variety of cancers or cancer in general.

Outside of tissue-based cancer diagnostics, most diagnostic methods rely on imaging techniques ranging from simple X-rays to MRIs and nuclear imaging, often using cancer- or tissue-targeted contrast agents to produce better images. However, even the most powerful imaging techniques cannot detect tumors smaller than about 2-5 mm in diameter. By the time a tumor has reached that size, it contains thousands of cells. Further, these sophisticated imagining techniques are too expensive to use during early stages of cancer, when the patient otherwise has no symptoms besides a small tumor that could easily be removed. Rather, complicated imaging diagnostics are most often reserved for patients who have had a large primary tumor and are suspected of having developed metastatic cancer. The small tumors detected are actually metastases produced as the cancer has spread. Thus, unlike primary tumors which often contain large numbers of benign cells, the small tumors detected contain thousands of malignant, metastatic cells, each of which is able to seed another tumor elsewhere in the body.

Clearly, detection of small metastatic tumors through current imaging techniques is really a last-ditch effort to save a critically ill patient. If these metastatic cells could be detected much earlier, such as when they first begin to travel through the blood, then a patient could begin receiving treatment for all of the metastatic tumors he or she would likely have while those tumors were still far too small to be detected by diagnostic imaging or any other current techniques. Thus a need exists for much earlier diagnosis of metastatic tumors, or detection of a greatly increased likelihood of metastatic tumors.

Yet another drawback in modern cancer diagnosis relates to its ability to be coupled to treatment. While some common mutations can be diagnosed through tissue samples and used to direct treatment somewhat specific for the patient's type of cancer, this approach is applicable for only a few types of cancer. Currently no diagnostic method is able to detect a wide range of types of cancer or to provide detailed targets for treatment in numerous types of cancer.

Finally, current cancer diagnostics, particularly those that rely upon tissue biopsies, are very poor at monitoring the progress or effectiveness of treatment. Thousands of dollars and possibly even patients' lives could be saved if treating physicians were able to tell when all or a substantial number of the cancer cells, or of a particular type of cancer cell have been eradicated. Additionally, by their nature cancer cells are able to change very rapidly. Thus, they may mutate even further during the course of a treatment, causing what was once a helpful drug to become powerless or harmful. In essence, the cancer cells may become resistant to the drug, much as bacteria become resistant to antibiotics. Cancer treatment would benefit greatly from diagnostic methods able to detect these and other changes that show the effectiveness of treatment or any further mutations of the patient's cancer cells.

### SUMMARY

The present invention relates to cancer markers, in particular a hyperset of markers for cancer generally and supsersets of markers for a specific type of cancer, as well as subsets of this hyperset and supersets.

The invention also relates to methods of screening blood or tissue using cancer detection reagents to detect cancer markers. Cancer detection reagents are short nucleic acids at least 17 bases in length having a specific sequence determined to correlate with the presence of cancer in a subject, but not with healthy tissue. Thus, the present invention relates to pathology-based diagnostics.

When blood is screened, it may be any type of blood, but to facilitate obtaining a sample, in most instances peripheral blood may be used. Although aspects of the present invention may be employed to detect cancer in a tissue, the descriptions here focus on peripheral blood due to the relative ease of obtaining a peripheral blood sample from a subject and its capacity to represent the cancer status of an entire animal, rather than a single tumor. However, it will be apparent to one skilled in the art how to adapt techniques designed for peripheral blood for use with other blood or tissues.

Cancer markers may include any mutation in the transcribed portions of the cellular DNA of a cell. These mutations may be detected through analysis based on the cancer cell's DNA or its mRNA using cancer detection reagents that correspond to the mutated DNA region, or cancer marker. In specific embodiments, PCR analysis, microarray analysis, or bead-based analysis may be used for cancer marker assays.

The cancer markers and corresponding cancer detection reagents were identified using proprietary software to examine databases of transcribed nucleic acid sequences from known cancers and cancer cell lines and to compare the sequences to the normal human transcriptome. Thus, these nucleic acid sequences represent mutations or abnormalities as compared to the transcriptome of humans without cancer. Specifically, the cancer markers are present in mRNA transcripts from cancer and universally absent in the entire healthy human transcriptome. Because the cancer markers only include transcribed sequences exclusive to cancer cells, they correspond to cancer-related mutations. Such mutations may include somatic mutations resulting in cancer, or they may also include rare abnormal variations present in the subject's genome.

Cancer detection reagents corresponding to these cancer markers, alone or in combination, may be used to determine the cancer marker profile of a subject. The cancer detection reagents may be used to detect cancer and to monitor the process of the cancer or of its treatment. Additionally, testing with the cancer detection reagents may be used to provide a cancer marker profile showing several mutations or abnormalities present in one or more metastatic cancer cells within the subject. Repeated testing can detect changes in the cancer marker profile of a subject, perhaps indicating the efficacy of treatment or the development of different metastatic cells.

In abundance among the cancer markers are sequences that repetitively occur in different cancer mRNA transcripts, thereby giving the cancer markers a one-to-many genetic association. This means one cancer detection reagent can detect multiple genes, each having the same cancer marker, and the detection is not dependent on the expression level of a single gene. The net result, both in-vitro and in-situ, is an enhanced detection capacity, facilitating detection even in samples having relatively low numbers of metastasized cancer cells.

All of the cancer markers will not be found in every cancer patient's blood or tumors. Instead, each patient will typically have a subset of the cancer markers present in their blood or tumors. Because many cancer markers are each associated with one or more genes, these subsets automatically produce genetic profiles that reflect the individuality of the patient's cancer.

In a specific embodiment, a general cancer diagnostic may be provided. Specifically, it has been determined that, while there are some variations in cancer markers among different types of cancer, some markers are very common in multiple types of cancer. Thus, a general diagnostic assay including these markers is provided. Such an assay may be particularly useful for routine screening or early diagnosis, when it is not known whether a subject has cancer, or the type of cancer the subject may have.

Additionally, cancer markers specific for certain types of cancer have been determined and ranked based on frequency of occurrence. For example, a subset of 59 markers frequently found in colon cancer have been located and used to create cancer detection reagents. Using these cancer type-specific sets of markers, diagnostic assays for a particular type of cancer are provided. These assays may be particularly useful in monitoring the progress or treatment of existing cancer. They may also be useful for routine diagnosis in subjects known to have a susceptibility to a particular type of cancer.

Finally, most cancer markers have been found in more than one gene. Thus, a diagnostic assay using a cancer detection reagent narrowly tailored to the cancer marker is very powerful in general cancer detection, but less useful in knowing which genes are affected. Knowledge of affected genes may affect the prognosis for or treatment of a patient. Thus, in yet another embodiment of the invention, gene-selective cancer detection reagents are provided. Such reagents are readily developed once a cancer marker has been identified. The cancer maker sequence may be located in a given gene, then flanking sequences found in the wild type gene may be included in a cancer detection reagent. Preferably, the flanking sequences included are of sufficient length to allow identification of the gene or genes having the cancer marker mutation in that subject, while remaining compatible with the type of assay being conducted.

Knowledge of the mutations present in a patient's cancer cells may be used in directing treatment. For example, drugs known to be effective against certain types of cancer or mutations in certain genes only may be prescribed or avoided based on the underlying mutations of a patient's cancer. Additionally, knowledge of patient-specific cancer mutations may be used to develop new classes of cancer drugs, including patient-specific cancer drugs targeted to the diagnosed mutations. These targeted drugs may affect the mutant proteins, particularly cell-surface proteins, or they may act on cellular nucleic acids, such as mRNA.

### BRIEF DESCRIPTION OF THE DRAWINGS

The present invention may be better understood through reference to the following detailed description taken in conjunction with the FIGUREs which illustrate various embodiments of the invention.

FIG. 1 illustrates several mutant cancer markers of the present invention found in the LTBR gene as compared to the sequence from healthy cell transcriptomes (SEQ ID NOS: 133-141). The location of a single nucleotide polymorphism (SNP) is indicated.

FIG. 2 illustrates a portion of an alignment between mRNA from four different cancer cell lines and four different cancer types, mapped to the corresponding healthy mRNA from 17 different genes (SEQ ID NOS: 7, and 142-158).

FIG. 3 illustrates a method of detecting a cancer marker. The common mutant DNA sequence shown is SEQ ID NO: 7.

FIG. 4 illustrates a sample cancer detection reagent (SEQ ID NOS 7 and 26).

FIG. 5 illustrates disparity in the presence of two common cancer markers between cancer cell lines (SEQ ID NOS: 9, and 27-29).

FIG. 6 illustrates correlation between individual cancer markers and cancer types.

FIG. 7 illustrates a method for PCR Reduction using cancer detection reagents.

FIG. 8 presents the results of PCR Reduction as analyzed on gels for cDNA from a healthy human and from tumor or blood samples of two cancer subjects.

FIG. 9 illustrates a method of blood testing and cancer marker profiling.

### DETAILED DESCRIPTION

The present invention relates to the detection of cancer, particularly metastatic cancer in a subject using an assay to detect cancer markers in samples from the subject. In a particular embodiment, detection may be accomplished using cancer detection reagents corresponding to the cancer markers.

The cancer detection reagents used in the present invention are presented primarily in the form of short DNA or other nucleic acid oligomers which correspond to cancer markers. These cancer markers have all been previously exhibited in cancerous tissue in a human. They may include mutations that imminently gave rise to the cancer, earlier mutations that likely increased the propensity for cancer, or abnormal allelic variants of a gene. Many are located in the transcribed portions of cellular DNA, particularly the exons of genes. However, cancer markers in accordance with the present invention may also correspond to other mutated DNA regions. Additionally, the markers may be detected in a sample using techniques that detect or amplify the mRNA or DNA in the sample. However, the markers may also be detected through assays for the peptides they encode, which may be predicted from the cancer marker sequences.

Cancer detection reagents may include both single-stranded and complementary double-stranded nucleic acids. The appropriate form of nucleic acids to use as a cancer detection reagent to identify a cancer marker will be apparent to one skilled in the art.

Identification of Cancer Markers

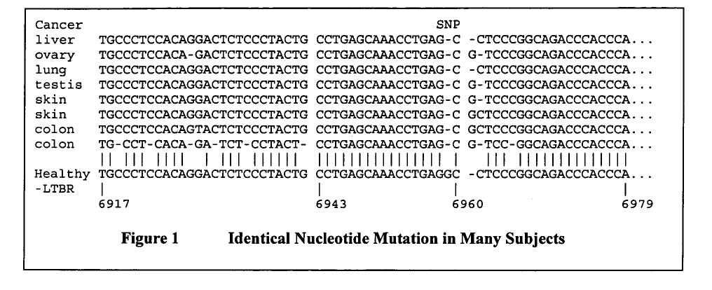

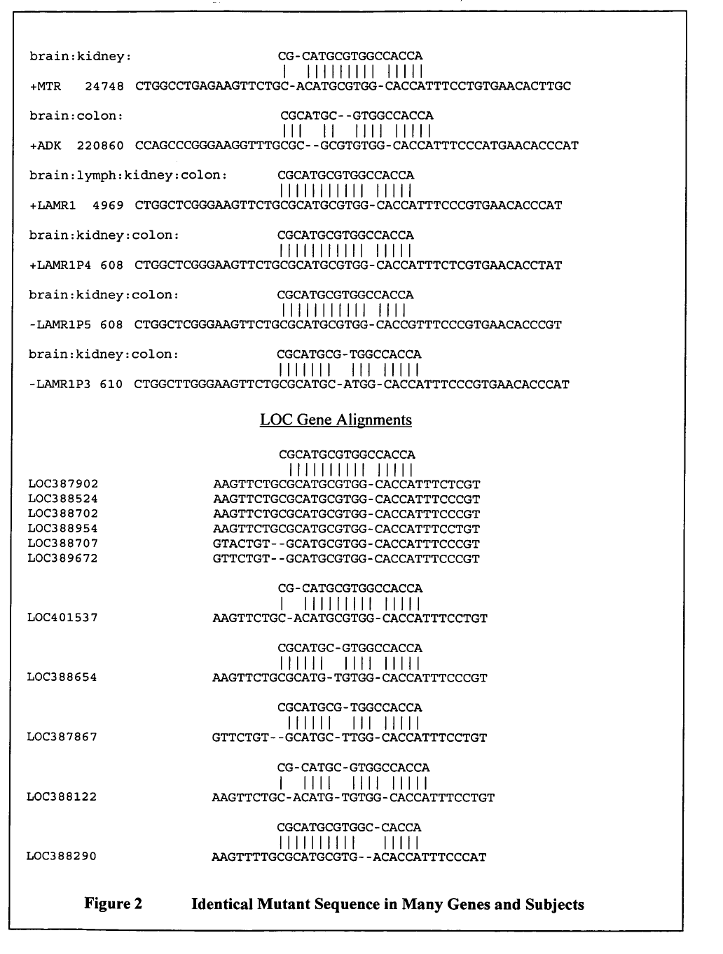

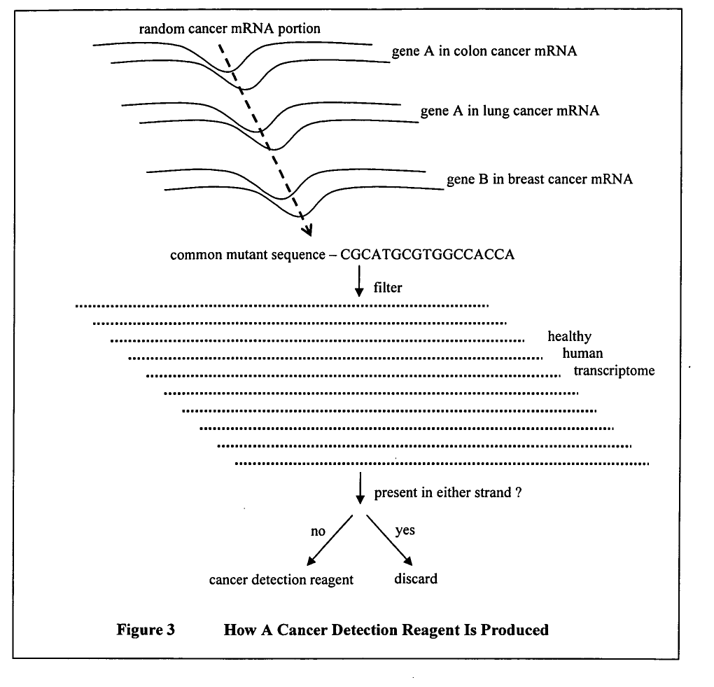

The cancer markers of the present invention were isolated using proprietary software and information from public databases recording genetic information about cancerous and healthy cells and tissues. Specifically, using proprietary software and supercomputers, random portions of mRNA data from cancer cell lines were compared to all the available mRNA data from all healthy cell lines, as diagramed in FIG. 3. This process yielded a database of cancer markers such as the two in FIG. 1 and FIG. 2.

The resultant database is referred to as the general cancer marker hyperset, which contains the sequences of hundreds of thousands of cancer markers, which may be embodied in cancer detection reagents of length 17-mer or greater, grouped into supersets according to cancer type. Each cancer marker in a superset must show up at least once in a cancer cell corresponding to the superset's cancer type. There is redundancy among the supersets because the cancer markers usually appear in supersets for many different cancer types.

The total number of cancer markers in the total cancer hyperset is constantly increased. Computer software currently runs non-stop, adding several thousand new cancer markers each month. Further, as new cancers arise, new cancer markers may be created. Based on currently available data, it is known that a superset for a single type of cancer may contain tens of thousands of cancer markers.

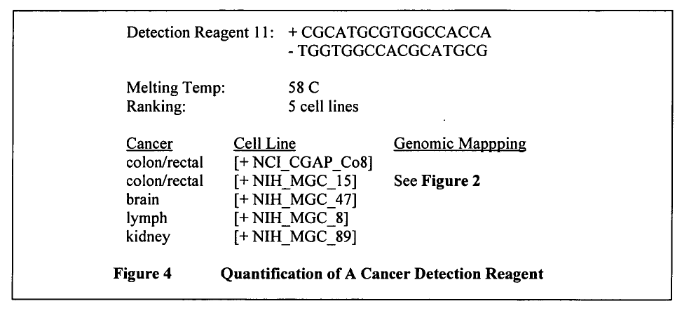

Because the cell lines used to isolate the mRNA molecules that contained the cancer markers are known and were derived from human subjects with cancer, it is possible to count these cell lines as past occurrences of the cancer markers in humans, as shown in FIG. 4. This yields a simple method for ranking the likelihood of occurrence of each cancer marker based on its past rate of occurrence in cancer cell lines.

Cancer markers represent a special kind of cancer mutation—one that has nucleic acid content exclusive to cancer cells. If such exclusivity were not present, the mutation would not be considered a cancer marker, as shown in FIG. 3. This condition in selecting cancer markers produces cancer detection reagents that detect useful differences in the genetics of cancer cells. This is an important criteria for diagnosing and treating cancer.

Muli-Gene Aspects

The cancer markers and detection reagents of the present invention are generally small and thus unsuitable for genomic mapping. However, the mRNA molecules containing the unisolated cancer markers can be mapped. In this manner, one may determine which genes are associated with each cancer marker.

Many genes may be associated with each cancer marker—the number of genes is normally in direct correlation to the number of unique mRNA molecules containing each cancer marker found in the public databases. Sometimes, hundreds of mRNA molecules in the databases contain a cancer marker, yielding hundreds of mapped genes. This is evident in TABLEs 1 and 2.

While many of the cancer markers are located in genes with no currently known relevance to cancer, some are located in genes known to be important in cancer. These cancer markers often represent SNPs, cryptic splicing and other genetic defects. For example, FIG. 1 illustrates a cancer detection reagent found in the Lymphotoxin Beta Receptor (LTBR) gene.

FIG. 1 shows that the same point mutation occurs in the same gene in different subjects with different types of cancer. Specifically, FIG. 1 shows a portion of an alignment between LTBR mRNA from eight different cancer cell lines and six different cancer types, mapped to the corresponding healthy LTBR mRNA. As the figure shows, the eight cancer LTBRs vary slightly between each other and the healthy LTBR. However at location 6959 bp, the cancer LTBRs vary identically, each missing a guanine (G) and yielding the same cancer marker, CCTGAGCAAACCTGAGC (SEQ ID NO: 6). This marker's presence in LTBR nucleic acids in a cell is an indicator of cancer's presence. This is a one-to-one genetic association.

FIG. 2 shows that the same cancer marker can result from different mutations in different genes, in different subjects with different types of cancer. Specifically, FIG. 2 shows a portion of an alignment between mRNA from four different cancer cell lines and four different cancer types, mapped to the corresponding healthy mRNA from 17 different genes. The mutations vary from gene to gene, but the net result is that the same cancer marker, CGCATGCGTGGCCACCA (SEQ ID NO: 7), is present in each gene. This marker's presence in each of the 17 genes is an indicator of cancer's presence in the corresponding cell or tissue. This sequence has a one-to-many genetic association.

The cancer markers shown in FIG. 1 and FIG. 2 are not dependent on any common functionality among the genes in which they appear or in the tissues in which these genes are expressed. Further, neither cancer marker has been found in the healthy human transcriptome. Therefore the presence of these markers in any mRNA transcript, not just those from genes shown in the figures, is an indicator of cancer's presence in the host cell. Because the sequences represent mRNAs exclusive to cancer cells, they reflect cancer-associated mutations. Also, if they are detected, one immediately knows which set of genes may contain them.

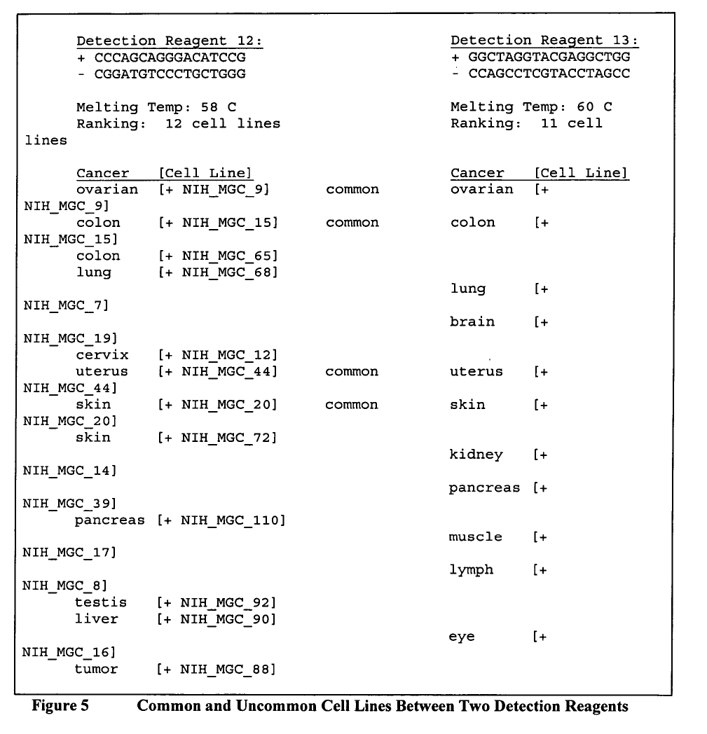

Cancer markers may be common to many genes and many cancers. This does not mean that every cancer marker will exist in every cancer cell line or cancer subject. This is demonstrated in FIG. 5 for two cancer markers and the cancer cell lines in which they occur.

Specific Subsets of Markers

Analysis of the cancer marker hyperset and supersets has revealed that a number of cancer markers are found frequently in a variety of different types of cancer. Thus these cancer markers may be identified as general cancer markers. General cancer markers have been identified and are included in TABLEs 1 and 2. These cancer markers were first identified as high frequency colon cancer markers and may also be used for that purpose.

TABLEs 1 and 2 lists the highest ranked 59 cancer markers in the colon cancer superset. These 59 cancer markers constitute a high frequency colon cancer marker subset. Associated genes are also indicated. Combined, there are over 1000 genes represented in the table. This means that the 59 colon cancer markers, when used in a detection capacity, can detect mutations in over 1000 genes—a sensitivity made possible by their one-to-many genetic association.

TABLE 1
Cancer Detection Reagents

| | | |
|---|---|---|
| ID | Candidate Apoptotic Sequence | Affected Cancers |

| | | |
|---|---|---|
| 5 | + GCCCAAGGAACCCCCTT | ovarian colorectal brain |
| | (SEQ ID NO: 21) | epid testis liver |
| | − AAGGGGGTTCCTTGGGC | |
| | (SEQ ID NO: 1) | |

Targeted Genes
CHCHD3(7) EEF1G(11) LOC136337(X) ABCC3(17)

| | | |
|---|---|---|
| 8 | + GCTCAGGTTTGCTCAGG | ovarian colorectal lung |
| | (SEQ ID NO: 22) | testis liver skin |
| | − CCTGAGCAAACCTGAGC | |
| | (SEQ ID NO: 6) | |

Targeted Genes
LTBR(12)

| | | |
|---|---|---|
| 9 | + TGTGCTTCTGGCAGGCC | breast colorectal brain |
| | (SEQ ID NO: 23) | adrenal eye |
| | − GGCCTGCCAGAAGCACA | |
| | (SEQ ID NO: 2) | |

Targeted Genes
GNB2L1(5)

| | | |
|---|---|---|
| 10 | + ACCTGGGATCCAGTTGGAGGACGGC | colorectal lung brain |
| | (SEQ ID NO: 24) | |
| | − GCCGTCCTCCAACTGGATCCCAGGT | |
| | (SEQ ID NO: 25) | |

Targeted Genes
ZNF500(16)

| | | |
|---|---|---|
| 11 | + CGCATGCGTGGCCACCA | colorectal brain lymph |
| | (SEQ ID NO: 7) | |
| | − TGGTGGCCACGCATGCG | |
| | (SEQ ID NO: 26) | |

Targeted Genes
LOC388707(1) LAMR1(3) LOC389672(8)

| | | |
|---|---|---|
| 12 | + CCCAGCAGGGACATCCG | ovarian colorectal lung |
| | (SEQ ID NO: 27) | cervix uterus skin |
| | − CGGATGTCCCTGCTGGG | pancreas testis liver |
| | (SEQ ID NO: 9) | |

Targeted Genes
MOV10(1)

| | | |
|---|---|---|
| 13 | + GGCTAGGTACGAGGCTGG | ovarian colorectal lung |
| | (SEQ ID NO: 28) | brain uterus skin |
| | − CCAGCCTCGTACCTAGCC | kidney pancreas muscle |
| | (SEQ ID NO: 29) | lymph eye |

Targeted Genes
AACS(12) AAMP(2) ABCF3(3) ACTB(7) ACTBP2(5) ACTG1(17)
ACTN1(14) ADCK4(19) ADPRT(1) AES(19) AFG3L2(18) AHSA1(14)
AIPL1(17) AKT1(14) ALDOA(16) ANAPC2(9) ANKRD19(9)
ANXA11(10) ANXA7(10) AP1M1(19) AP2A1(19) AP2M1(3) APCL(19)
APOE(19) ARHGDIA(17) ARHGEF1(19) ARHGEF16(1) ARL6IP4(12)
ARPC2(2) ASPH(8) 11ASRGL1(11) ASS(9) ATF4(22) ATF5(19)
ATP1A1(1) ATP5A1(18) ATP5F1(1) ATP5O(21) AUTL2(X) AZ2(3)
bA395L14.12(2) BAT3(6) BCAS3(17) BLP1(8) BRMS1(11) BSG(19)
BTF3(5) C10orf45(10) C14orf12G(14) C20orf41(20) 2orf17(2)
C3orf4(3) C4orf9(4) C5orfG(5) CG.1A(X) C6orf107(6)
6orf11(6) CGorf4S(6) C7orf30(7) CACNA2D3(3) CAMKK2(12)
CASP4(11) CASQ1(1) CBS(21) CBX7(22) CBX8(17) CCND3(6)
CCT3(1) CCT5(5) CCTGA(7) CCT7(2) 0D74(5) CD79A(19)
CD79B(17) CDC20(1) CDC2L2(1) CDCA5(11) CDCA8(1) CDH12(5)
CDH24(14) CDIPT(1G) CDK4(12) CDW92(9) CEECAM1(9) CENPB(20)
CGI-96(22) CHCHD3(7) CIDEB(14) CNOT10(3) COMT(22) ORO1A(16)
CORO2A(9) COTL1(16) CRN(4) CRTAP(3) CRYBB2P1(22) CS(12)
CTAG3(6) CYB5-M(16) DBH(9) DBI(2) DCLRE1C(10) DCTN2(12)
DDB1(11) DDX1O(11) DDX56(7) DCCR8(22) DGKA(12) DHCR24(1)
DKFZp434B227(3) DKFZP434C171(5) DKFZP434K04G(16)
DKFZP564D172(5) DKFZp564K142(X) DKFZp58GM1819(8) DNAJB1(19)
DNCH1(14) DNM2(19) DRIM(12) DustyPK(1) E1B-AP5(19) E2F4(16)
EDARADD(1) EEF1D(8) EEF1G(11) EEF2(19) EIF2B5(3) EIF2S1(14)
eIF3k(19) EIF3S1(15) EIF3S2(1) ETF3S5(11) EIF3S7(22)
EIF3SB(16) ETF3S9(7) EIF4G1(3) ELMO2(20) ENDOG(9) ENO1(1)
ENO1P(1) ENTPD8(17) EPAC(12) ETEDH(4) FAH(15) FAM31B(1)
FANCA(1G) FBL(19) FBXO7(22) FDFT1(8) FECH(18) FGFR4(5)
FKBP1B(2) EKBP8(19) FKSG17(8) FLT1(11) FLJ00038(9)
FLJ10241(19) FLJ12750(12) FLJ12875(1) FLJ14800(12)
FLJ14827(12) FLJ20071(18) FLJ20203(1) FLJ20294(11)
ELJ20487(11) FLJ21827(11) FLJ22028(12) FLJ22688(19)
ELJ25222(15) FLJ27099(14) FLJ31121(5) FLJ32452(12)
FLJ35827(11) FLJ38464(9) FLJ44216(5) FMN2(1) FMO5(1)
FOSL1(11) FSCN1(7) FUS(16) G22P1(22) G2AN(11) GA17(11)
GALK2(15) GAPD(12) GCC1(7) GCDH(19) GDI2(10) GA1(22)
GGCX(2) GIT1(17) GLUL(1) GNB2L1(5) GOLGB1(3) GPAA1(8)
GPI(19) GRHPR(9) GRSF1(4) GSPT1(16) GSTM4(1) GYS1(19)
H3F3B(17) HAND1(5) HARS2(20) HAX1(1) HCA127(X) HCCR1(12)
HCG4(6) HDAC1(1) HDLBP(2) HLA-B(6) HMGA1(6) HMGA1L3(12)
HMGN1(21) HMGN2(1) HNRPD(4) HNRPH3(10) HNRPU(1) HPS4(22)
HRMT1L1(21) HS3ST4(16) HSA97G1(5) HSPA9B(5) HSPB1(7)
HSPC142(19) HSPC242(22) HSPCB(6) HSPCP1(4) HSPD1(2) 1D3(1)
IER3(6) IGFBP4(17) IGHV4-34(14) L1RL1LG(19) ILF2(1)
ILVBL(19) IMPDH2(3) ITGB4BP(20) JIK(12) JM4(X) K-ALPHA-
1(12) KCNN2(5) KCTD1(18) KHSRP(19) KIAAO141(5) KIAAO182(16)
KIAA0258(9) KIAA0582(2)KIAA0774(13) KIAA1O49(16)
KIAA1055(15) KTAA1115(19) KIAA1211(4) KIAA1765(3) KNS2(14)
KPNB1(17) KRT17(17) KRT5(12) KRT8(12) LAMR1P3(14)
LARGE(22) LASP1(17) LCP1(13) LDHB(12) LDHBP(X) LENG5(19)
LGALS1(22) LGALS3BP(17) LIMK2(22) L1N28(1) LMO7(13)
LOC113174(11) LOC127253(1) LOC129138(22) LOC136337(x)
LOC137829(1) LOC144581(12) LOC145414(14) LOC145989(15)
LOC146253(16) LOC148640(1) LOC149501(1) LOC150417(22)
LOC158078(9) LOC192133(14) LOC201292(17) LOC220717(2)
LOC221838(7) LOC253482(9) LOC266724(2) LOC266783(1)
LOC283747(15) LOC283820(16) LOC284089(17) L00284393(19)
LOC285214(3) LOC285741(6) L00285752(6) LOC286444(X)
LOC339395(1) LOC339799(2) LOC342705(18) LOC348180(16)
LOC374443(12) LOC387703(10) LOC388076(15) LOC388344(117)
LOC388519(19) LOC388556(19) LOC388642(1) LOC388654(1)
LOC388968(2) LOC389181(3) LOC389240(4) LOC389342(5)
LOC389849(X) LOC389901(X) LOC390415(13) LOC390814(17)
LOC390860(18) LOC391634(4) LOC391717(4) LOC391739(5)
LOC391800(5) LOC399942(11) LOC399969(11) LOC4000GB(12)
LOC400586(17) LOC400634(17) LOC400744(1) LOC400954(2)
LOC400963(2) LOC4O1010(2) LOC401146(4) LOC401245(G)
LOC401316(7) LOC401677(11) LOC401838(1G) LOC402057(22)
LOC402142(3) LOC402259(7) LOC402579(7) LOC402650(7)
LOC51149(5) LOC91272(5) LOC92755(8) LPPR2(19) LSP1(11)
LU(19) LY6E(8) MGPRBP1(19) MAGED1(X) MAMDC2(9) MAP3K4(6)
MAPRE1(20) MARS(12) MBD3(19) MCM2(3) MECP2(X) MESDC1(15)
MFGE8(15) MGAT4B(5) MGC10540(17) MGC10986(17) MCC11061(2)
MGC12966(7) MGC19764(17) MGC20446(11) MGC2601(16)
MGC2714(11) MGC2749(19) MGC29816(8) MGC3162(12) MGC35555(8)
MGC4606(16) MGC48332(5) MGC52000(2) MGC5508(11)
MGC71999(17) MGST2(4) MRPL2(G) MRPL28(16) MRPL9(1)
MRPS12(19) MRPS27(5) MRPS34(16) MSH3(5) MSHG(2) MSN(X)
MSNL1(5) MIJS81(11) MVP(16) MYBL2(20) MYCT1(6) NACA(12)
NAP1L1(12) NARF(17) NARS(18) NCOA4(10) NDE1(16) NDUFA10(2)
NDUFAB1(16) NDUFB9(8) NDUFS1(2) NDUFS2(1) NICE-3(1) NICE-
4(1) NME1(17) NME3(16) NONO(X) NPM1(5) NQO2(6) NRBF2(10)
NRBP(2) NS(3) NUDT8(11) NUP210(3) NUTF2(16) NUTF2P2(14)
NXF1(11) OAZ1(19) OK/SW-c1.56(6) OS-9(12) OSBPL9(1) PBP(12)
PCCA(13) PCOLCE2(3) PDAP1(7) PDHA1(X) PDXP(22) PEA15(1)
PECI(G) Pfs2(16) PGD(1) PGK1(X) PH-4(3) PHGDH(1) PIGT(20)
PIK4CA(22) PKD1P3(16) PKM2(15) PKM2(15) PLEKHA4(19) PM5(16)
PMM2(16) POLDIP3(22) POLE3(9) POLH(6) POLR2E(19) POLR2H(3)
POU2E1(1) PPFIBP2(11) PPIE(1) PPOX(1) PPP1R15A(19)
PPP1R8(1) PPP2R1A(19) PPP4C(16) PRAME(22) PRDX1(1)
PRKACA(19) PRNPIP(1) PR01855(17) PRPF31(19) PSAP(10)
PSMC2(7) PSMD2(3) PSME1(14) PSPC1(13) PTBP1(19) PTPN6(12)
PTPRCAP(11) PTPRD(9) PTPRG(3) PTTG1IP(21) PYCR1(17)
RAB32(6) RAE1(20) RALGDS(9) RAN(12) RANP1(6) RARS(5)
RASAL1(12) RBBP7(X) RDH11(14) REC14(15) RER1(1) RFC2(7)
RGS16(1) RHEBL1(12) RIOK1(6) RNF10(12) RNF20(9) RNF8(6)
RoXaN(22) RPL1O(X) RPL10P1(21) RPL13(16) RPL14(3) RPL15(3)
RPL15P2(14) RPL24(3) RPL28(19) RPL3(22) RPL30(8) RPL35(9)
RPL35A(3) RPL37A(2) RPL37AP1(20) RPL5(1) RPL8(8) RPL9(4)
RPLP0(12) RPLP0P2(11) RPLP2(11) RPS10(6) RPS14(5) RPS15(19)
RPS16(19) RPS17(15) RPS17P2(5) RPS19(19) RPS19P1(20)
RPS2(16) RPS20(8) RPS20P3(5) RPS2L1(20) RPS3(11) RPSG(9)
RPS9(19) RPS9P2(22) RRP4(9) RRP40(9) RTKN(2) RUVBL1(3)
RUVBL2(19) S100A1G(1) SAFB(19) SARS(1) SART3(12) SATB1(3)
SBDS(7) SCD(10) SCYL1(11) SEC31L1(4) SFRS2(17) SH2D3A(19)
SH3BP1(22) SH3BP5(3) SHMT2(12) SIAHBP1(8) SIN3A(15)
SKB1(14) SLC25A3(12) SLC25A6(X) SLC25A6(Y) SLC7A5(16)
SMARCA4(19) SMARCB1(22) SNRPA(19) SNRPA1(15) SNRPB(20)
SNRPC(6) SNX17(2) SNXG(14) SOD1(21) SPINT1(15) SPPL2B(19)
SRP14(15) ST7(7) STAG3(7) STAMBP(2) STARD7(2) STAT6(12)
STIM1(11) STK33(11) STMN1(1) STXBP2(19) SUPT1GH(14)
SUPT5H(19) SV2A(1) SV2C(5) TADA2L(17) TADA3L(3) TAF11(6)
TAGLN2(1) TCEB1(8) TCL1A(14) TD-60(1) TDPX2(9) TTC(2)
Tino(19) TIP120A(12) TK1(17) TMEM4(12) TMSB4X(X) TOR3A(1)
TPI1(12) TPK1(7) TPM3(1) TRAP1(16) TRAPPC1(17) TRAPPC3(1)
TRBC2(7) TRIP10(19) TRP14(17) TUBA3(12) TUBAG(12) TUBB2(9)
TUSC2(3) TXNDC5(6) TXNTP(1) UBAP2(9) UBC(12) UBE2J2(1)
USP11(X) USP7(16) VAMP8(2) VWF712) VWFP(22) WAC(10)
WBSCR1(7) WDR1(4) WDR18(19) WDR34(9) XPNPEP1(10) XPO5(6)
YAP(1) YKT6(7) YWHAB(20) ZNF212(7) ZNF24(18)
ZNF41(X)ZNF44(19) ZNE574(19) ZSWIM6(5)

| | | |
|---|---|---|
| 14 | + CGCTGGTGTTAATCGGCCGAGG | ovarian colorectal lung |
| | (SEQ ID NO: 30) | brain uterus skin |
| | − CCTCGGCCGATTAACACCAGCC | kidney pancreas muscle |
| | (SEQ ID NO: 31) | lymph eye |

Targeted Genes
ARHGDIA(17) ATP7A(X) BTE3(5) CAD(2) CD59(l1) CLNS1A(1l)
CSNK2B(6) DAP3(1) DHTKD1(l0) DNAJB12(10) FBL(19)
FLJ22688(19) GPT(8) H2AFX(11) HDLBP(2) HSPB1(7) INSM1(20)
JIK(12) LOC129138(22) LOC144483(12) LOC145414(14)
LOC158078(9) LOC221838(7) LOC285752(6) LOC286444(X)
LOC389912(X) LOC401146(4) LOC51149(5) LOC83468(12) MSHG(2)
NFAT5(16) NME2(17) RPL3(22) RPS2L1(20) SDBCAG84(20)
SDCCAG3(9) SH3BPl(22) SMARCA4(19) WHSC2(4) XPO5(6)
ZSWIM6(5)

| | | |
|---|---|---|
| 15 | + GGGGGTGAATCGGCCGAGG | ovarian colorectal lung |
| | (SEQ ID NO: 32) | brain uterus skin |
| | − CCTCGGCCGATTCACCCCC | kidney pancreas muscle |
| | (SEQ ID NO: 33) | lymph eye |

Targeted Genes
ACTB(7) ANKRD19(9) ASB1(2) ATF4(22) C1orf26(1) CHGB(20)
COG1(17) CPS1(2) CPT1A(11) CX3CL1(16) CYFIP2(5) ELKS(12)
FMO5(1) ETL(19) G2AN(l1) GFPT1(2) GNB2L1(5) GOT2(16)
GTF3C5(9) HCAl27(X) HSPA4(5) HSPA8(1l) HSPCB(6) HSPCP1(4)
ILVBL(19) KDELR1(19) KIAA1917(17) LAPTM4B(8) LOC116166(15)
LOC126037(19) LOC138198(9) LOC143920(11) LOC158714(X)
LOC283820(16) LOC340600(X) LOC388783(20) LOC390730(16)
LOC391044(1) LOC391634(4) LOC392437(X) LOC401308(7)
LOC401677(11) LOC402461(7) LOC84549(8) LOC90850(16) LYN(8)
MAP4(3) NCL(2) NICE-3(1) NICE-4(1) NJMU-R1(17) NONO(X)
ODC1(2) PHB(17) PKD1P3(16) PKM2(15) PM5(16) PRNPIP(1)
PTPN11(12) RCN1(11) RGS4(1) RNP8(6) RPL5(1) RPN1(3)
S100A11(1) SAE1(19) SCAMP3(1) SLC25A3(12) SORD(15) ST7(7)
TIMM50(19) TM4SE11(16) U5-116KD(17) UBE2G2(21) UCHL1(4)
VARS2(6) WDR6(3) ZNF160(19)

| | | |
|---|---|---|
| 16 | + GCTGGGTGTGAATCGGCCGAGC | ovarian colorectal lung |
| | (SEQ ID NO: 34) | brain uterus skin |
| | − CCTCGGCCGATTCACACCCAGC | kidney pancreas muscle |
| | (SEQ ID NO: 35) | lymph eye |

Targeted Genes
ABCB6(2) ACTB(7) ARHGEF1(19) ATP5G2(12) AZ2(3) BAT3(6)
BCL2L14(12) BID(22) C14orf94(14) C6orf49(6) Cab45(1)
CBX7(22) CDK4(12) CHCHD2(7) CHCHD3(7) CNOT7(8) COX5B(2)
DKFZP761D0211(16) DMAP1(1) DNPEP(2) EDARADD(1) EML2(19)
ENDOG(9) ENO1(1) ENO1P(1) FGFR4(5) FLJ11773(12)
FLJ13868(16) FLJ22169(2) FTL(19) FCJS(16) G22P1(22)
GOLGA3(12) HDLBP(2) HH114(15) HIC2(22) HLA-B(6) HSPCA(14)
HSPCB(6) HSPCP1(4) HSRNAFEV(2) ILKAP(2) IMPDH2(3) IRX4(5)
ITGA1(5) K-ALPHA-1(12) KIAA0195(17) LDHB(12) LIG1(19)
LOC128439(20) LOC130617(2) LOC134147(5) LOC136337(x)
LOC220717(2) LOC285741(6) LOC387703(1O) LOC388783(20)
LOC3891169(3) LOC389181(3) LOC389424(6) LOC389787(9)
LOC389901(X) LOC391634(4) LOC392437(X) LOC392647(7)
LOC399942(11) LOC400006(12) LOC4011316(7) LOC402057(22)
LOC402579(7) LOC90321(19) LOC90850(16) LYRIC(8) MACF1(1)
MAPT(17) MGC13170(19) MGC4549(19) MRPL23(11) MVP(16)
NIF1E14(19) OSGEP(14) PA2G4(12) PDIP(16) PELO(5) PEX10(1)
PKD1-1ike(1) PKM2(15) POFUT1(20) PREP(6) PRKAB1(12)
PSMD3(17) PTMA(2) RPL13A(19) RPLP0(12) RPLP0P2(11)
RPS11(19) RPS17(15) RPS17P2(5) RPS3(11) SH3YL1(2)
SLC25A19(17) SNRPA(19) SNRPC(6) SPTAN1(9) SUPT5H(19)
SYNGR2(17) TH1L(20) TIMM50(19) TPM3(1) TPT1(13) TRAF4(17)
TRIM29(11) TUBA3(12) TUBA6(12) TUFM(16) UPK3B(7) UQCRH(1)
WBSCR1(7) WDR18(19) WDR34(9)

| | | |
|---|---|---|
| 17 | + AGGTACGAGGCCGGGTGTT | ovarian colorectal lung |
| | (SEQ ID NO: 36) | brain uterus skin |
| | − AACACCCGGCCTCGTACCT | kidney pancreas muscle |
| | (SEQ ID NO: 37) | lymph eye |

Targeted Genes
ANXA2(15) ANXA2P1(4) ANXA2P2(9) AP4E1(15) ARF3(12) ATF4(22)
ATP1A1(1) ATP5A1(18) AUTL2(X) BANP(16) C20orf43(20)
C6orf69(6) CCT3(1) CCT7(2) CDT6(1) CHCHD3(7) CLDN2(X)
CLECSF9(12) CTAG3(6) DKC1(X) E2F4(16) EEF1G(11) EIF3S8(16)
EST1B(1) FLJ10349(1) FLJ10871(8) FLJ32370(8) FRAP1(1)
FSCN1(7) GAPD(12) GNPAT(1) HMOX1(22) HNRPF(10) K-ALPHA-
1(12) KIAA1917(17) KRT18(12) LOC136337(X) LOC145414(14)
LOC158345(9) LOC284393(19) LOC285752(6) LOC339395(1)
LOC388975(2) LOC389181(3) LOC389342(5) LOC389849(X)
LOC399942(11) LOC400966(2) LOC401369(7) LOC92755(8)
LOC92755(8) LOC94431(16) M96(1) MAP3K13(3) MGAT4B(5)
MRPL48(11) MRPL48P1(6) NFE2L1(17) NIFU(12) NIPSNAP1(22)
OK/SW-cl.56(6) P4HB(17) PCDH11X(X) PFKM(12) PITRM1(10)
PKM2(15) RNPC4(14) RPL18(19) RPL3(22) RPLPOP2(11)
RPS17P2(5) RPS3(11) RPS5(19) RRN3(16) RYK(3) SEC24A(5)
SLC25A3(12) SOD1(21) STRN4(19) TINF2(14) TM9SE4(20)
TRIM2(4) TUBA3(12) TUBAG(12) TUBB2(9) UQCRC1(3) WBP1(2)
YARS(1) YKT6(7) ZFP106(15) ZSWIM6(5)

| | | |
|---|---|---|
| 18 | + GTGTTAATCGGCCGAGG | ovarian colorectal lung |
| | (SEQ ID NO: 38) | brain uterus skin |
| | − CCTCGGCCGATTAACAC | kidney pancreas muscle |
| | (SEQ ID NO: 39) | lymph eye |

Targeted Genes
ABCF2(7) ABHD3(18) ACOXL(2) ACTB(7) ACTG1(17) ADCYG(12)
ADRM1(20) AK2(1) AK3(1) ANP32B(9) ANXA2P2(9) ARF4L(17)
ARG2(14) ARHC(1) ARHGDIA(17) ARPC1B(7) ARPC2(2) ARRB2(17)
ASPH(8) ATP5B(12) ATP7A(X) BACH(1) BANP(16) BAZ1A(14)
BGN(X) BID(22) BLP1(8) BTF3(5) C14orf94(14) C20orf35(20)
C22orf5(22) CAD(2) CAP1(1) CAPNS1(19) CARM1(19) CASP4(11)
CASQ1(1) CCT3(1) CD59(11) CDK2(12) CHCHD3(7) CLDN2(X)
CLECSF9(12) CLNS1A(11) CNOT7(8) COMT(22) COQ6(14) CPE(4)
CSNK2B(6) CTSB(8) CYB5-M(16) DAP3(1) DAXX(6) DBH(9) DCI(16)
DDOST(1) DDR1(6) DDX42(17) DHCR24(1) DHTKD1(10)
DJ159A19.3(1) DKPZp434B227(3) DKFZP586J0619(7) DNAJA1(9)
DNAJB12(10) DND1(5) E2F1(20) EDARADD(1) EEF1D(8) EEF1G(11)
E124(11) EIF2BS(3) EIF3S61P(22) EIF3S8(16) EMD(X) ENO1(1)
ENO1P(1) ENO2(12) EPLIN(12) ESD(13) EXT2(11) FBL(19)
FBXO7(22) FLJ10597(1) FLJ11822(17) FLJ12541(15)
FLJ12949(19) FLJ21103(11) FLJ22688(19) FLJ22843(X)
FLJ27099(14) FLJ34836(5) PLNA(X) FSCN1(7) FTL(19) FTS(16)
GAPD(12) GBF1(10) GCN5L2(17) GGA2(16) GOLGA3(12) GOSR2(17)
GPR17(2) GPT(8) GUSB(7) GYS1(19) H2AFX(11) H3F3B(17)
HADHA(2) HADHAP(4) HDGF(1) HDLBP(2) HMOX2(16) HNRPAB(5)
HNRPDL(4) HNRPU(1) HOXA9(7) HRB2(12) HRIHFB2122(22)
HS2ST1(1) HSPB1(7) HSPCA(14) HSPCAL2(4) HSPCAL3(11)
IDH3B(20) 1F130(19) 1L411(19) IMPDH2(3) IMUP(19) INSIG1(7)
TNSM1(20) ISYNA1(19) JARTD1A(12) JIK(12) JMJD2B(19) JRK(8)
JUNB(19) K-ALPHA-1(12) KHSRP(19) KIAA0182(16) KIAA0582(2)
KIAA0738(7) KIAA1614(1) KIAA1952(9) KPNB1(17) KRT17(17)
KRT19(17) KRT7(12) KRT8(12) LDHB(12) LDHBP(X) LIMR(12)
LIMS2(2) LMNA(1) L00113444(1) LOC115509(1G) LOC129138(22)
LOC136337(X) LOC144483(12) LOC145414(14) LOC145767(15)
LOC146053(15) LOC149501(1) LOC153027(4) LOC158078(9)
LOC158473(9) LOC192133(14) LOC220433(13) LOC221838(7)
LOC256000(4) LOC283820(16) LOC285741(G) LOC285752(6)
LOC286444(X) LOC339395(1) LOC339736(2) LOC341056(11)
LOC387851(12) LOC388076(15) LOC388524(19) LOC388642(1)
LOC388707(1) LOC388783(20) LOC388907(22) LOC388975(2)
LOC389912(X) LOC390819(17) LOC392437(X) LOC392647(7)
LOC399942(11) LOC399994(12) LOC400397(15) LOC400631(17)
LOC400879(22) LOC400966(2) LOC401146(4) LOC401308(7)
LOC401316(7) LOC401426(7) LOC401504(9) LOC401972(1)
LOC401987(1) LOC402461(7) LOC402618(7) LOC51149(5)
LOC83468(12) LOC90313(17) LOC92755(8) LSM4(19) LTBP3(11)
LYPLA2(1) MAGED1(X) MAP1LC3B(16) MAP2K1(15) MBD3(19)
MCM5(22) MCMG(2) MESDC2(15) MGC11335(16) MGC19595(19)
MGC20446(11) MGC2714(11) MGC35182(9) MIR16(16) MRPL12(17)
MRPL41(9) MRPL45(17) MRPS26(20) MSHG(2) MYBL2(20)
NAP1L1(12) NCSTN(1) NDUFA9(12) NF1(17) NFAT5(16)
NIPSNAP1(22) NME1(17) NME2(17) NONO(X) NPEPPS(17) NUDT5(10)
NUPG2(19) OK/SW-cl.56(6) ORC6L(16) P2RY6(11) PDLTM1(10)
PEA15(1) PEF(1) PFKM(12) PFKP(10) PGK1(X) PGK1P2(19)
PIK4CA(22) PITRM1(10) PKM2(15) PM5(16) PMM2(16) POLR3D(8)
PPAP2C(19) PPM1G(2) PPP1CA(11) PPT1(1) PQLC1(18) PRDX4(X)
PR01855(17) PROCR(20) PRSS15(19) PSMC3(11) PSMC3P(9)
PSMC4(19) PTOV1(19) QDPR(4) RAB8A(19) RABEP1(17) RAC1(7)
RAC4(X) RAE1(20) RARS(5) REC14(15) RELA(11) RNF10(12)
RNF2G(11) RNPS1(16) RPL22(1) RPL3(22) RPL35A(3) RPL5(1)
RPL8(8) RPLP2(11) RPN2(20) Rpp25(15) RPS2(16) RPS2L1(20)
RPS3A(4) RPS4X(X) RPS5(19) RPS6KB2(11) RRM2(2) RRM2P3(X)
RSHL1(19) S100A1G(1) SAE1(19) SARS(1) SDBCAG84(20)
SDCCAG3(9) SDHB(1) SF3B3(16) SF4(19) SH3BP1(22) SIN3A(15)
SLC25AG(X) SLC25AG(Y) SLC41A3(3) SLC43A1(11) SMARCA4(19)
SNRPN(15) SOX10(22) SPARC(5) SPINT1(15) SRPRB(3) STRN4(19)
SUPT5H(19) TAGLN2(1) TCOF1(5) TEAD2(19) THOC3(5)
TIMELESS(12) TM4SF8(15) TM9SF4(20) TMEM4(12) TNIP1(5)
TPI1(12) TPT1(13) TRAP1(16) TUBA1(2) TUBA3(12) TUBAE(12)
U5-116KD(17) UBA2(19) UBE1(X) UCHL1(4) UPK3B(7) UQCRC1(3)
VASP(19) VCP(9) V1P32(10) WBP1(2) WBSCR1(7) WDR1(4)
WHSC2(4) XPO5(6) YARS(1) ZDHHC12(9) ZDHHC16(10) ZNF313(20)
ZNE559(19) ZNF584(19) ZSWIM6(5)

| | | |
|---|---|---|
| 19 | + AGATGGGTACCAACTGT | ovarian colorectal lung |
| | (SEQ ID NO: 40) | brain pancreas |
| | − ACAGTTGGTACCCATCT | muscle testis eye |
| | (SEQ ID NO: 41) | |

Targeted Genes
LOC220717(2) RPLP0P2(11) RPLP0(12)

| | | |
|---|---|---|
| 20 | + CGGCTAGGTACGAGGCTGGGGT | ovarian colorectal lung |
| | (SEQ ID NO: 42) | brain uterus skin |
| | − ACCCCAGCCTCGTACCTAGCCG | kidney muscle lymph eye |
| | (SEQ ID NO: 43) | |

Targeted Genes
C5orf6(5) CASQ1(1) CCT3(1) CORO2A(9) CTAG3(6)
ENTPD8(17) FLNA(X) FOSL1(11) GAPD(12) HSPC171(16)
HSPCB(6) HSPCP1(4) KIAAO29G(16) LOC388556(19)
LOC389849(X) LOC391634(4) MBTPS1(16) NARE(17) NONO(X)
PEA15(1) RER1(1) RIOK1(G) RPS3(11) RPS9(19) RPS9P2(22)
SATB1(3) SLC12A4(16) TADA3L(3) ZNF44(19)

| | | |
|---|---|---|
| 21 | + GAGGCGGGTGTGAATCGGCCGAGG | ovarian colorectal brain |
| | (SEQ ID NO: 44) | uterus skin |
| | − CCTCGGCCGATTCACACCCGCCTC | pancreas muscle lymph eye |
| | (SEQ ID NO: 45) | |

Targeted Genes
ACTG1(17) ATP5G3(2) CCT6A(7) CN2(18) CORO1A(16) FTL(19)
HMGA1(6) HSPCB(G) HSPCP1(4) LMAN2(5) LOC257200(2)
LOC388783(20) LOC391634(4) LOC392437(X) MGC16824(16)
MGC5178(16) NASP(1) NASPP1(8) PEDN5(12) PME-1(11)
RAB5C(17) SPTAN1(9) TERF2IP(16) UBB(17) UBBP4(17) UQCR(19)

| | | |
|---|---|---|
| 22 | + AGGTACGAGGCCGGTGT | ovarian colorectal brain |
| | (SEQ ID NO: 46) | uterus skin |
| | − ACACCGGCCTCGTACCT | kidney pancreas muscle |
| | (SEQ ID NO: 47) | lymph |

Targeted Genes
ALDH1A1(9) ARPC2(2) ATP5A1(18) BST2(19) CD79B(17) DBH(9)
DDB1(11) EIF2B5(3) EIF3SGIP(22) EIF3S6IPP(14) ELF3(1)
ENO1(1) FLJ27099(14) G22P1(22) G6PD(X) GAPD(12)
GTF3C1(16) KIAA1068(7) KIAA1068(7b) KIAA1952(9)
LOC145414(14) LOC192133(14) LOC285741(6) LOC346085(6)
LOC387703(10) LOC387922(13) LOC388076(15) LOC389849(X)
LOC389901(X) LOC92755(8) MCM7(7) MCSC(9) MRPL45(17)
NASP(1) NASPP1(8) NDST2(10) OAZ1(19) OK/SW-cl.56(6)
RPL18(19) RPS8(1) TAGLN2(1) TPT1(13) XRCC1(19)
ZNF271(18) ZSWIM6(5)

| | | |
|---|---|---|
| 23 | + GTTAATCGGCCGAGGCGC | ovarian colorectal lung |
| | (SEQ ID NO: 48) | brain uterus skin |
| | − GCGCCTCGGCCGATTAAC | kidney pancreas muscle |
| | (SEQ ID NO: 49) | lymph |

Targeted Genes
CSNK2B(6) EIF3SGIP(22) INSIG1(7) KIAA1115(19) KRT7(12)
LOC401658(11) LOC402057(22) LOC89958(9) LOC92755(8)
MGC3047(1) OK/SW-cl.56(6) PROCR(20) RAN(12) RPS17(15)
RPS17P2(5) SMT3H1(21) UPP1(7) WHSC2(4)

| | | |
|---|---|---|
| 24 | + AGACCAACAGAGTTCGG | ovarian colorectal lung |
| | (SEQ ID NO: 50) | skin kidney pancreas |
| | − CCGAACTCTGTTGGTCT | |
| | (SEQ ID NO: 51) | |

Targeted Genes
novel mapping

| | | |
|---|---|---|
| 25 | + TGGCTTCGTGTCCCATGCA | breast ovarian colorectal |
| | (SEQ ID NO: 52) | lung skin muscle |
| | − TGCATGGGACACGAAGCCA | liver |
| | (SEQ ID NO: 53) | |

Targeted Genes
GAPD(12) GAPDL4(4) KIAA0295(15) KLHL8(4) LOC389849(X)

| | | |
|---|---|---|
| 26 | + CCGGCTGTAAATCGGCCGA | ovarian colorectal brain |
| | (SEQ ID NO: 54) | uterus skin |
| | − TCGGCCGATTTACACCCGG | pancreas muscle lymph |
| | (SEQ ID NO: 55) | |

Targeted Genes
C19orf13(19) EIF3SGP1(6) EIF3SG(8) GNB2L1(5) GTF2H3(12)
HDAC1(1) HSPCA(14) KRT5(12) PAK1IP1(6) PD2(19) QARS(3)
SFRS10(3)

| | | |
|---|---|---|
| 27 | + GCCGGTGTGAATCGGCCGA | colorectal lung brain |
| | (SEQ ID NO: 56) | uterus skin kidney |
| | − TCGGCCGATTCACACCGGC | pancreas muscle |
| | (SEQ ID NO: 57) | |

Targeted Genes
ARHC(1) ATP7B(13) BCAP31(X) C20orf35(20) CTDSP2(12)
EBNA1BP2(1) FLJ10737(1) FLJ20254(2) G22P1(22) HDLBP(2)
HMGN2(1) HS3ST4(16) H5A272196(17) HSPC117(22) LCP1(13)
LOC339395(1) LOC387703(10) LOC389901(X) MGC11242(17)
MRPL51(12) NAP1L1(12) NDUFV1(11) POLDIP2(17) PSMB1(6)
SIRT2(19) SQSTM1(5) SRPR(11) STK25(2) SV2C(5) TAGLN2(1)
TJP1(15) XRCC1(19)

| | | |
|---|---|---|
| 28 | + TCATGATGGTGTATCGATGA | ovarian colorectal lung |
| | (SEQ ID NO: 58) | brain skin bone |
| | − TCATCGATACACCATCATGA | |
| | (SEQ ID NO: 59) | |

Targeted Genes
JIK(12) LOC400963(2) LOC91561(11) LOC286444(X)

| | | |
|---|---|---|
| 29 | + GCTCGGTGTTAATCGGCCGA | ovarian colorectal brain |
| | (SEQ ID NO: 60) | uterus skin |
| | − TCGGCCGATTAACACCGAGC | pancreas lymph eye |
| | (SEQ ID NO: 61) | |

Targeted Genes
CASP4(11) GGA2(16) HRIHFB2122(22) INSIG1(7) KHSRP(19)
LOC388642(1) LOC400879(22) PRDX4(X) RPS2(16) SDHB(1)
SLC25A6(X) SLC25A6(Y) TPI1(12) TRAP1(16) V1P32(10)

| | | |
|---|---|---|
| 30 | + TGGGGTTAATCGGCCGAGG | ovarian colorectal lung |
| | (SEQ ID NO: 62) | uterus skin |
| | − CCTCGGCCGATTAACCCCA | pancreas lymph eye |
| | (SEQ ID NO: 63) | |

Targeted Genes
ADRBK1(11) BCKDK(16) LOC220717(2) MGC3329(17) MRPL15(8)
QARS(3) RPLPO(12) RPLP0P2(11) RPS9(19) RPS9P2(22)
SPATA11(19) SRM(1) TADA3L(3) TUEM(16)

| | | |
|---|---|---|
| 31 | + AGGCCGGTGTTAATCGGCCGA | ovarian colorectal lung |
| | (SEQ ID NO: 64) | brain uterus skin |
| | − TCGGCCGATTAACACCCGCCT | kidney pancreas lymph |
| | (SEQ ID NO: 65) | |

Targeted Genes
ACTG1(17) AK3(1) ANXA2P2(9) ARPC2(2) ATP5B(12) CPE(4)
DBH(9) DCI(16) DHCR24(1) DJ159A19.3(1) EEF1D(8) ENO1(1)
GOLGA3(12) HADHA(2) HADHAP(4) HIP-55(7) HNRPU(1) JMJD2B(19)
K-ALPHA-1(12) K1AA1952(9) LOC145414(14) LOC158473(9)
LOC285741(6) LOC387851(12) LOC388524(19) LOC388707(1)
LOC392647(7b) LOC399942(11) LOC399994(12) LOC401316(7)
LOC401504(9) LOC401987(1) MRPL45(17) NF1(17) NME1(17)
PRSS15(19) RABEP1(17) SOX10(22) SRPRB(3) TAGLN2(1) TPT1(13)
TUBA3(12) TUBAG(12) VCP(9) WBSCR1(7) ZSWIMG(5)

| | | |
|---|---|---|
| 32 | + TGGTGAATCGGCCGAGGGT | ovarian colorectal brain |
| | (SEQ ID NO: 66) | uterus skin |
| | − ACCCTCGGCCGATTCACCA | kidney pancreas lymph |
| | (SEQ ID NO: 67) | |

Targeted Genes
ACADS(12) C20orf149(20) DCTN3(9) DPYSL3(5) EIF3S1(15)
IPO4(14) KIAA0152(12) LOC388556(19) LOC401092(3) PRDX5(11)
PSMF1(20) RAB11A(15) RPL10(X) RPS9(19) RPS9P2(22)
STXBP2(19) ZNF3(7) ZNF-U69274(3)

| | | |
|---|---|---|
| 33 | + AGCAAGTATGACAACAGC | colorectal lung cervix |
| | (SEQ ID NO: 68) | skin pancreas muscle |
| | − GCTGTTGTCATACTTGCT | |
| | (SEQ ID NO: 69) | |

Targeted Genes
GAPD(12) LOC389849(X)

| | | |
|---|---|---|
| 34 | + CTTAAACCAAGCTAGCC | colorectal prostate brain |
| | (SEQ ID NO: 70) | skin bone testis |
| | − GGCTAGCTTGGTTTAAG | eye |
| | (SEQ ID NO: 71) | |

Targeted Genes
LOC143371(10) LOC150554(2) LOC158383(9) YWHAZ(8)

| | | |
|---|---|---|
| 35 | + CAGTCTACATCACGTGG | colorectal lung cervix |
| | (SEQ ID NO: 72) | brain kidney lymph |
| | − CCACGTGATGTAGACTG | liver eye |
| | (SEQ ID NO: 73) | |

Targeted Genes
LOC359792(Y) LOC400039(12) PCDH11X(X) PCDH11Y(Y)

| | | |
|---|---|---|
| 36 | + AATCTCCTGTTACACTCA | ovarian colorectal brain |
| | (SEQ ID NO: 74) | epid testis |
| | − TGAGTGTAACAGGAGATT | |
| | (SEQ ID NO: 75) | |

Targeted Genes
LOC146909(17)

| | | |
|---|---|---|
| 37 | + GCCCAAGGAACCCCCTT | ovarian colorectal lung |
| | (SEQ ID NO: 76) | skin testis liver eye |
| | − AAGGGGGTTCCTTGGGC | |
| | (SEQ ID NO: 77) | |

Targeted Genes
ABCC3(17) CHCHD3(7) EEF1G(11) LOC136337(X)

| | | |
|---|---|---|
| 38 | + GGCTAGGACGAGGCCGGG | colorectal brain skin |
| | (SEQ ID NO: 78) | kidney pancreas |
| | − CCCGGCCTCGTCCTAGCC | muscle lymph |
| | (SEQ ID NO: 79) | |

Targeted Genes
ATPGV1E1(22) CCT4(2) CHGB(20) DHX9(1) EIF3S8(16)
LOC343515(1) MAP2K2(19) NDUPA9(12) NDUFA9P1(22) SCARB1(12)

| | | |
|---|---|---|
| 39 | + GAGAAGGTTCCCGGGAA | colorectal lung pancreas |
| | (SEQ ID NO: 80) | lymph liver eye |
| | − TTCCCGGGAACCTTCTC | |
| | (SEQ ID NO: 81) | |

Targeted Genes
CHCHD3(7) EEF1G(11) LOC136337(X) MGC10471(19)

| | | |
|---|---|---|
| 40 | + GTGTTACTCGGCCGAGG | colorectal lung brain |
| | (SEQ ID NO: 82) | uterus skin kidney |
| | CCTCGGCCGAGTAACAC | pancreas muscle |
| | (SEQ ID NO: 83) | |

Targeted Genes
ACLY(17) ADAR(1) ALDH1A1(9) C12orf10(12) GNAI2(3) K-ALPHA-
1(12) LMNB2(19) LOC400671(19) PPIE(1) RYK(3) TTYH3(7)
TUBA3(12) TUBA6(12)

| | | |
|---|---|---|
| 41 | + TTGAATCGGCCGAGGGTG | ovarian colorectal lung |
| | (SEQ ID NO: 84) | brain pancreas |
| | − CACCCTCGGCCGATTCAA | muscle eye |
| | (SEQ ID NO: 85) | |

Targeted Genes
CTNP(14) COTL1(16) FLJ39075(16) GNB2L1(5) KRT19(17)
KRT4(12) LOC92305(4) MCSC(9)
PCNT1(17) PH-4(3) RPL8(8) ZNF337(20)

| | | |
|---|---|---|
| 42 | + GCCGGGTGGTGAATCGG | ovarian colorectal brain |
| | (SEQ ID NO: 86) | uterus skin kidney |
| | − CCGATTCACCACCCGGC | muscle |
| | (SEQ ID NO: 87) | |

Targeted Genes
ACTG1(17) CHCHD3(7) DFFA(1) DPYSL3(5) PRDX5(11) SYMPK(19)
TSPAN-1(1) ZDHHC16(10)

| | | |
|---|---|---|
| 43 | + GCCGGTCGTTAATCGGC | colorectal lung brain |
| | (SEQ ID NO: 88) | uterus skin kidney |
| | − GCCGATTAACCACCGGC | pancreas |
| | (SEQ ID NO: 89) | |

Targeted Genes
C6orf109(6) CFL1(11) FLJ30934(11) GALNT2(1) K-ALPHA-1(12)
LOC145414(14) LOC285752(6) LOC399942(11) LOC56931(19)
PCDH18(4) PSMC3(11) RPL3(22) SARS(1) STK19(6) TCF7L1(2)
TETRAN(4) TUBA3(12) TUBA6(12)

| | | |
|---|---|---|
| 44 | + GGGCGCAGCGACATCAG | colorectal prostate lung |
| | (SEQ ID NO: 90) | adrenal pancreas |
| | − CTGATGTCGCTGCGCCC | lymph eye |
| | (SEQ ID NO: 91) | |

Targeted Genes
TREX2(X)

| | | |
|---|---|---|
| 45 | + GCTATTAGCAGATTGTGT | colorectal lung kidney |
| | (SEQ ID NO: 92) | muscle testis eye |
| | − ACACAATCTGCTAATAGC | |
| | (SEQ ID NO: 93) | |

Targeted Genes
LOC399942(11) K-ALPHA-1(12) TUBA3(12) TUBAG(12)

| | | |
|---|---|---|
| 46 | + TGTTAATCTCCTGTTACACTCA | ovarian colorectal brain |
| | (SEQ ID NO: 94) | epid testis liver |
| | − TGAGTGTAACAGGAGATTAACA | |
| | (SEQ ID NO: 95) | |

Targeted Genes
LOC146909(17)

| | | |
|---|---|---|
| 47 | + CCACCGCACCGTTGGCC | ovarian colorectal cervix |
| | (SEQ ID NO: 96) | skin kidney testis |
| | − GGCCAACGGTGCGGTGC | |
| | (SEQ ID NO: 97) | |

Targeted Genes
FBXW5(9)

| | | |
|---|---|---|
| 48 | + ACCTGGAGCCCTCTGAT | colorectal lung skin |
| | (SEQ ID NO: 98) | kidney muscle liver |
| | − ATCAGAGGGCTCCAGGT | |
| | (SEQ ID NO: 99) | |

Targeted Genes
LOC399942(11) K-ALPHA-1(12) TUBA3(12) TUBA6(12)

| | | |
|---|---|---|
| 49 | + TCAGACAAACACAGATCG | colorectal prostate lung |
| | (SEQ ID NO: 100) | brain muscle |
| | − CGATCTGTGTTTGTCTGA | |
| | (SEQ ID NO: 101) | |

Targeted Genes
LOC285900(7) DGKI(7) LOC402525(7b) LOC388460(18) RPLG(12)

| | | |
|---|---|---|
| 50 | + GAGAATACTGATTGAGACCTA | ovarian colorectal skin |
| | (SEQ ID NO: 102) | kidney lymph testis |
| | − TACGTCTCAATCAGTATTCTC | |
| | (SEQ ID NO: 103) | |

Targeted Genes
LOC92755(8) OK/SW-cl.56(6)

| | | |
|---|---|---|
| 51 | + CCAGCCAGCACCCAGGC | colorectal gall skin |
| | (SEQ ID NO: 104) | pancreas lymph |
| | − GCCTGGGTGCTGGCTGG | |
| | (SEQ ID NO: 105) | |

Targeted Genes
ATP5A1(18) FLJ10101(9) IL9R(X) IL9R(Y) LOC392325(9)
LOC400481(16) RELA(11)

| | | |
|---|---|---|
| 52 | + TAGACCAACAGAGTTCC | colorectal lung skin |
| | (SEQ ID NO: 106) | kidney muscle liver |
| | − GGAACTCTGTTGGTCTA | |
| | (SEQ ID NO: 107) | |

Targeted Genes
novel mapping

| | | |
|---|---|---|
| 53 | + CTAGGTACGAGGCTGGGTTTT | colorectal lung uterus |
| | (SEQ ID NO: 108) | skin muscle lymph |
| | − AAAACCCAGCCTCGTACCTAG | |
| | (SEQ ID NO: 109) | |

Targeted Genes
ACTG1(17) LOC81691(16) PSAP(10) SFRS2(17)

| | | |
|---|---|---|
| 54 | + CGAGGCGGGTGTTAATCGGCC | colorectal lung brain |
| | (SEQ ID NO: 110) | skin pancreas |
| | − GGCCGATTAACACCCGCCTCG | lymph eye |
| | (SEQ ID NO: 111) | |

Targeted Genes
ACTB(7) ADCYG(12) BID(22) EIF3S6IP(22) EIF3S8(16) K-ALPHA-
1(12) MRPL12(17) PDLIM1(10) RARS(5) RPN2(20) S100A1G(1)
TUBA1(2)

| | | |
|---|---|---|
| 55 | + AAGGCTAGGTAGAGGCTG | ovarian colorectal brain |
| | (SEQ ID NO: 112) | pancreas muscle eye |
| | − CAGCCTCTACCTAGCCTT | |
| | (SEQ ID NO: 113) | |

Targeted Genes
ANP32B(9) C20orf14(20) CAD(2) COL14A1(8) CTNNBL1(20)
DOK4(16) ENO1(1) FLJ22301(1) HSPCB(6) HSPCP1(4) K-ALPHA-
1(12) LOC339395(1) LOC391634(4) LOC400397(15) PKM2(15)
RACGAP1(12) STATIP1(18) VASP(19)

| | | |
|---|---|---|
| 56 | + CATGGCCATGCTGTGCA | colorectal uterus skin |
| | (SEQ ID NO: 114) | testis |
| | − TGCACAGCATGGCCATG | |
| | (SEQ ID NO: 115) | |

Targeted Genes
DNPEP(2) MATP(5)

| | | |
|---|---|---|
| 57 | + AGGTACGAGGCCGGTGTTAATCGGCCGA | ovarian colorectal lung |
| | (SEQ ID NO: 116) | brain kidney lymph |
| | − TCGGCCGATTAACACCGGCCTCGTACCT | |
| | (SEQ ID NO: 117) | |

Targeted Genes
ARPC2(2) DBH(9) ENO1(1) K1AA1952(9) LOC145414(14)
LOC285741(6) MRPL45(17) TAGLN2(1) TPT1(13) ZSWIMG(5)

| | | |
|---|---|---|
| 59 | + TGCTGCCCTCAATGGTC | colorectal lung cervix |
| | (SEQ ID NO: 118) | skin muscle eye |
| | − GACCATTGAGGGCAGCA | |
| | (SEQ ID NO: 119) | |

Targeted Genes
novel mapping

| | | |
|---|---|---|
| 60 | + AGGCCGGTGGTTAATCGGCCGAGG | colorectal brain uterus |
| | (SEQ ID NO: 120) | skin kidney pancreas |
| | − CCTCGGCCGATTAACCACCGGCCT | |
| | (SEQ ID NO: 121) | |

Targeted Genes
C6orf109(6) GALNT2(1) LOC145414(14) LOC285752(6)
LOC56931(19) PCDH18(4) PSMC3(11) RPL3(22) STK19(6)
TETRAN(4)

| | | |
|---|---|---|
| 61 | + GAGGCCGGTGGTTAATCGGCCGAG | colorectal brain uterus |
| | (SEQ ID NO: 122) | skin kidney pancreas |
| | − CTCGGCCGATTAACCACCGGCCTC | |
| | (SEQ ID NO: 123) | |

Targeted Genes
C6orf109(6) LOC145414(14) LOC285752(6) LOC56931(19)
PCDH18(4) PSMC3(11) RPL3(22)
STK19(6) TETRAN(4)

| | | |
|---|---|---|
| 62 | + GCTAGGTACGAGGCTGGGTTTT | colorectal lung uterus |
| | (SEQ ID NO: 124) | skin muscle lymph |
| | − AAAACCCAGCCTCGTACCTAGC | |
| | (SEQ ID NO: 125) | |

Targeted Genes
ACTG1(17) PSAP(10) SERS2(17)

| | | |
|---|---|---|
| 63 | + AACATACGGCTAGGTACGA | ovarian colorectal brain |
| | (SEQ ID NO: 126) | uterus lymph eye |
| | − TCGTACCTAGCCGTATGTT | |
| | (SEQ ID NO: 127) | |

Targeted Genes
CIZ1(9) FLJ20203(1) FLJ23416(17) MGC3162(12) MSF(17)
SWAP70(11) YAP(1)

| | | |
|---|---|---|
| 64 | + GGTGGTAATCGGACGAGG | colorectal lung brain |
| | (SEQ ID NO: 128) | uterus skin muscle |
| | − CCTCGTCCGATTACCACC | |
| | (SEQ ID NO: 129) | |

Targeted Genes
AKT1(14) CHGA(14) CHRNA3(15) EMS1(11) FLJ20244(19)
FLJ22169(2) GNB2L1(5) LOC130617(2) L00284393(19)
LOC347422(X) LOC388642(1) LOC389342(5) SLC4A2(7) TIMM17B(X)
TPI1(12) YKT6(7)

| | | |
|---|---|---|
| 65 | + GGGTGATCGGACGAGGC | ovarian colorectal lung |
| | (SEQ ID NO: 130) | brain pancreas eye |
| | − GCCTCGTCCGATCACCC | |
| | (SEQ ID NO: 131) | |

Targeted Genes
ACTG1(17) ANKRD19(9) DNAJB11(3) EEF1D(8) HSPCA(14)
HSPCAL2(4) HSPCAL3(11)
LOC126037(19) LOC399704(6) RABAC1(19)

| | | |
|---|---|---|
| 66 | + ACATGCCTAGGGTTCAA | colorectal lung cervix |
| | (SEQ ID NO: 132) | pancreas testis eye |
| | − TTGAACCCTAGGCATGT | |
| | (SEQ ID NO: 5) | |

Targeted Genes
EEF1A1(6) LOC401146(4)

TABLE 2

Colon Cancer Marker Subset and Primers
(SEQ ID NOS:159-344)

| | | |
|---|---|---|
| 3 | Cancer Oligo: | + CCTCTGTTAATCTCCTGTTACA |
| | | − TGTAACAGGAGATTAACAGAGG |
| | Primer Oligo: | + CCTCTGTTAATCTCCTGTT |
| | | − AACAGGAGATTAACAGAGG |
| | Reference: | Tissue: colon_14528_155 |
| | | Cancer Temp: 62° C. Primer Temp: 54° C. |
| 5 | Cancer Oligo: | + GCCCAAGGAACCCCCTT |
| | | − AAGGGGGTTCCTTGGGC |
| | Reference: | Tissue: colon_52909_1157 |
| | | Cancer Temp: 56° C. |
| 6 | Cancer Oligo: | + GACTGAATGCACCCAATATCCGACCTGGCTGCGTGT |
| | | − ACACGCAGCCAGGTCGGATATTGGGTGCATTCAGTC |
| | Primer Oligo: | + GACTGAATGCACCCAATAT |
| | | − ATATTGGGTGCATTCAGTC |
| | Reference: | Tissue: colon_7084_373 |
| | | Cancer Temp: 112° C. Primer Temp: 54° C. |
| 7 | Cancer Oligo: | + CACCCTCTGTTAATCTCCTGTTACA |
| | | − TGTAACAGGAGATTAACAGAGGGTG |
| | Primer Oligo: | + CACCCTCTGTTAATCTCC |
| | | − GGAGATTAACAGAGGGTG |
| | Reference: | Tissue: colon_14528_154 |
| | | Cancer Temp: 72° C. Primer Temp: 54° C. |
| 8 | Cancer Oligo: | + GCTCAGGTTTGCTCAGG |
| | | − CCTGAGCAAACCTGAGC |
| | Reference: | Tissue: colon_22882_6 |
| | | Cancer Temp: 54° C. |
| 9 | Cancer Oligo: | + TGTGCTTCTGGCAGGCC |
| | | − GGCCTGCCAGAAGCACA |
| | Reference: | Tissue: colon_46693_132 |
| | | Cancer Temp: 56° C. |
| 10 | Cancer Oligo: | + ACCTGGGATCCAGTTGGAGGACGGC |
| | | − GCCGTCCTCCAACTGGATCCCAGGT |
| | Primer Oligo: | + ACCTGGGATCCAGTTGG |
| | | − CCAACTGGATCCCAGGT |
| | Reference: | Tissue: colon_38162_1403 |
| | | Cancer Temp: 82° C. Primer Temp: 54° C. |
| 11 | Cancer Oligo: | + CGCATGCGTGGCCACCA |
| | | − TGGTGGCCACGCATGCG |
| | Reference: | Tissue: colon_41436_2209 |
| | | Cancer Temp: 58° C. |
| 12 | Cancer Oligo: | + CCCAGCAGGGACATCCG |
| | | − CGGATGTCCCTGCTGGG |
| | Reference: | Tissue: colon_134925_1443 |
| | | Cancer Temp: 58° C. |
| 13 | Cancer Oligo: | + GGCTAGGTACGAGGCTGG |
| | | − CCAGCCTCGTACCTAGCC |
| | Primer Oligo: | + GGCTAGGTACGAGGCTG |
| | | − CAGCCTCGTACCTAGCC |
| | Reference: | Tissue: colon_121812_797 |
| | | Cancer Temp: 60° C. Primer Temp: 56° C. |
| 14 | Cancer Oligo: | + GGCTGGTGTTAATCGGCCGAGG |
| | | − CCTCGGCCGATTAACACCAGCC |
| | Primer Oligo: | + GGCTGGTGTTAATCGGC |
| | | − GCCGATTAACACCAGCC |
| | Reference: | Tissue: colon_122287_1352 |
| | | Cancer Temp: 72° C. Primer Temp: 54° C. |
| 15 | Cancer Oligo: | + GGGGGTGAATCGGCCGAGG |
| | | − CCTCGGCCGATTCACCCCC |
| | Primer Oligo: | + GGGGGTGAATCGGCCG |
| | | − CGGCCGATTCACCCCC |
| | Reference: | Tissue: colon_122308_1392 |
| | | Cancer Temp: 66° C. Primer Temp: 56° C. |
| 16 | Cancer Oligo: | + GCTGGGTGTGAATCGGCCGAGG |
| | | − CCTCGGCCGATTCACACCCAGC |
| | Primer Oligo: | + GCTGGGTGTGAATCGGC |
| | | − GCCGATTTCACACCCAGC |
| | Reference: | Tissue: colon_123371_2691 |
| | | Cancer Temp: 74° C. Primer Temp: 56° C. |
| 17 | Cancer Oligo: | + AGGTACGAGGCCGGGTGTT |
| | | − AACACCCGGCCTCGTACCT |
| | Primer Oligo: | + AGGTACGAGGCCGGGT |
| | | − ACCCGGCCTCGTACCT |
| | Reference: | Tissue: colon_124205_4458 |
| | | Cancer Temp: 62° C. Primer Temp: 54° C. |
| 18 | Cancer Oligo: | + GTGTTAATCGGCCGAGG |
| | | − CCTCGGCCGATTAACAC |
| | Reference: | Tissue: colon_124503_5628 |
| | | Cancer Temp: 54° C. |
| 19 | Cancer Oligo: | + AGATGGGTACCAACTGT |
| | | − ACAGTTGGTACCCATCT |
| | Reference: | Tissue: colon_132239_12738 |
| | | Cancer Temp: 50° C. |
| 20 | Cancer Oligo: | + CGGCTAGGTACGAGGCTGGGGT |
| | | − ACCCCAGCCTCGTACCTAGCCG |
| | Primer Oligo: | + CGGCTAGGTACGAGGC |
| | | − GCCTCGTACCTAGCCG |
| | Reference: | Tissue: colon_124479_5522 |
| | | Cancer Temp: 74° C. Primer Temp: 54° C. |
| 21 | Cancer Oligo: | + GAGGCGGGTGTGAATCGGCCGAGG |
| | | − CCTCGGCCGATTCACACCCGCCTC |
| | Primer Oligo: | + GAGGCGGGTGTGAATCG |
| | | − CGATTCACACCCGCCTC |
| | Reference: | Tissue: colon_124382_5017 |
| | | Cancer Temp: 82° C. Primer Temp: 56° C. |
| 22 | Cancer Oligo: | + AGGTACGAGGCCGGTGT |
| | | − ACACCGGCCTCGTACCT |
| | Reference: | Tissue: colon_124545_5835 |
| | | Cancer Temp: 56° C. |
| 23 | Cancer Oligo: | + GTTAATCGGCCGAGGCGC |
| | | − GCGCCTCGGCCGATTAAC |
| | Primer Oligo: | + GTTAATCGGCCGAGGCG |
| | | − CGCCTCGGCCGATTAAC |
| | Reference: | Tissue: colon_124554_5891 |
| | | Cancer Temp: 60° C. Primer Temp: 56° C. |
| 24 | Cancer Oligo: | + AGACCAACAGAGTTCGG |
| | | − CCGAACTCTGTTGGTCT |
| | Reference: | Tissue: colon_128799_3222 |
| | | Cancer Temp: 52° C. |
| 25 | Cancer Oligo: | + TGGCTTCGTGTCCCATGCA |
| | | − TGCATGGGACACGAAGCCA |
| | Primer Oligo: | + TGGCTTCGTGTCCCATG |
| | | − CATGGGACACGAAGCCA |
| | Reference: | Tissue: colon_128901_3427 |
| | | Cancer Temp: 60° C. Primer Temp: 54° C. |
| 26 | Cancer Oligo: | + CCGGGTGTAAATCGGCCGA |
| | | − TCGGCCGATTACACCCGG |
| | Primer Oligo: | + CCGGGTGTAAATCGGCC |
| | | − GGCCGTTTACACCCGG |
| | Reference: | Tissue: colon_121791_713 |
| | | Cancer Temp: 62° C. Primer Temp: 56° C. |
| 27 | Cancer Oligo: | + GCCGGTGTGAATCGGCCGA |
| | | − TCGGCCGATTCACACCGGC |
| | Primer Oligo: | + GCCGGTGTGAATCGGC |
| | | − GCCGATTCACACCGGC |
| | Reference: | Tissue: colon_122271_1321 |
| | | Cancer Temp: 64° C. Primer Temp: 54° C. |
| 28 | Cancer Oligo: | + TCATGATGGTGTATCGATGA |
| | | − TCATCGATACACCATCATGA |
| | Reference: | Tissue: colon_122810_2119 |
| | | Cancer Temp: 56° C. |
| 29 | Cancer Oligo: | + GCTCGGTGTTAATCGGCCGA |
| | | − TCGGCCGATTAACACCGAGC |
| | Primer Oligo: | + GCTCGGTGTTAATCGGC |
| | | − GCCGATTAACACCGAGC |
| | Reference: | Tissue: colon_123361_2652 |
| | | Cancer Temp: 64° C. Primer Temp: 54° C. |
| 30 | Cancer Oligo: | + TGGGGTTAATCGGCCGAGG |
| | | − CCTCGGCCGATTAACCCCA |
| | Primer Oligo: | + TGGGGTTAATCGGCCGA |
| | | − TCGGCCGATTAACCCCA |
| | Reference: | Tissue: colon_123408_2783 |
| | | Cancer Temp: 62° C. Primer Temp: 54° C. |
| 31 | Cancer Oligo: | + AGGCCGGTGTTAATCGGCCGA |
| | | − TCGGCCGATTAACACCGGCCT |
| | Primer Oligo: | + AGGCCGGTGTTAATCGG |
| | | − CCGATTAACACCGGCCT |
| | Reference: | Tissue: colon_124428_5212 |
| | | Cancer Temp: 68° C. Primer Temp: 54° C. |
| 32 | Cancer Oligo: | + TGGTGAATCGGCCGAGGGT |
| | | − ACCCTCGGCCGATTCACCA |
| | Primer Oligo: | + TGGTGAATCGGCCGAGG |
| | | − CCTCGGCCGATTCACCA |
| | Reference: | Tissue: colon_124548_5844 |
| | | Cancer Temp: 62° C. Primer Temp: 56° C. |
| 33 | Cancer Oligo: | + AGCAAGTATGACAACAGC |
| | | − GCTGTTGTCATACTTGCT |
| | Reference: | Tissue: colon_124841_107 |
| | | Cancer Temp: 52° C. |
| 34 | Cancer Oligo: | + CTTAAACCAAGCTAGCC |
| | | − GGCTAGCTTGGTTTAAG |
| | Reference: | Tissue: colon_125327_1240 |
| | | Cancer Temp: 50° C. |
| 35 | Cancer Oligo: | + CAGTCTACATCACGTGG |
| | | − CCACGTGATGTAGACTG |
| | Reference: | Tissue: colon_131175_9725 |
| | | Cancer Temp: 52° C. |
| 36 | Cancer Oligo: | + AATCTCCTGTTACACTCA |
| | | − TGAGTGTAACAGGAGATT |
| | Reference: | Tissue: colon_131332_10159 |
| | | Cancer Temp: 50° C. |
| 37 | Cancer Oligo: | + GCCCAAGGAACCCCCTT |
| | | − AAGGGGGTTCCTTGGGC |
| | Reference: | Tissue: colon_52909_1157 |
| | | Cancer Temp: 56° C. |
| 38 | Cancer Oligo: | + GGCTAGGACGAGGCCGGG |
| | | − CCCGGCCTCGTCCTAGCC |
| | Primer Oligo: | + GGCTAGGACGAGGCCG |
| | | − CGGCCTCGTCCTAGCC |
| | Reference: | Tissue: colon_121817_833 |
| | | Cancer Temp: 64° C. Primer Temp: 56° C. |
| 39 | Cancer Oligo: | + GAGAAGGTTCCCGGGAA |
| | | − TTCCCGGGAACCTTCTC |
| | Reference: | Tissue: colon_123283_2553 |
| | | Cancer Temp: 54° C. |
| 40 | Cancer Oligo: | + GTGTTACTCGGCCGAGG |
| | | − CCTCGGCCGAGTAACAC |
| | Reference: | Tissue: colon_123389_2740 |
| | | Cancer Temp: 56° C. |
| 41 | Cancer Oligo: | + TTGAATCGGCCGAGGGTG |
| | | − CACCCTCGGCCGATTCAA |
| | Reference: | Tissue: colon 124408 5119 |
| | | Cancer Temp: 58° C. |
| 42 | Cancer Oligo: | + GCCGGGTGGTGAATCGG |
| | | − CCGATTCACCACCCGGC |
| | Reference: | Tissue: colon_124566_5929 |
| | | Cancer Temp: 58° C. |
| 43 | Cancer Oligo: | + GCCGGTGGTTAATCGGC |
| | | − GCCGATTAACCACCGGC |
| | Reference: | Tissue: colon_124579_5999 |
| | | Cancer Temp: 56° C. |
| 44 | Cancer Oligo: | + GGGCGCAGCGACATCAG |
| | | − CTGATGTCGCTGCGCCC |
| | Reference: | Tissue: colon_128875_3358 |
| | | Cancer Temp: 58° C. |
| 45 | Cancer Oligo: | + GCTATTAGCAGATTGTGT |
| | | − ACACAATCTGCTAATAGC |
| | Reference: | Tissue: colon_130347_7716 |
| | | Cancer Temp: 50° C. |
| 46 | Cancer Oligo: | + TGTTAATCTCCTGTTACACTCA |
| | | − TGAGTGTAACAGGAGATTAACA |
| | Primer Oligo: | + TGTTAATCTCCTGTTACACT |
| | | − AGTGTAACAGGAGATTAACA |
| | Reference: | Tissue: colon_131332_10158 |
| | | Cancer Temp: 60° C. Primer Temp: 54° C. |
| 47 | Cancer Oligo: | + CCACCGCACCGTTGGCC |
| | | − GGCCAACGGTGCGGTGG |
| | Primer Oligo: | + CCACCGCACCGTTGGC |
| | | − GCCAACGGTGCGGTGG |
| | Reference: | Tissue: colon_131939_11900 |
| | | Cancer Temp: 60° C. Primer Temp: 56° C. |
| 48 | Cancer Oligo: | + ACCTGGAGCCCTCTGAT |
| | | − ATCAGAGGGCTCCAGGT |
| | Reference: | Tissue: colon_132839_14455 |
| | | Cancer Temp: 54° C. |
| 49 | Cancer Oligo: | + TCAGACAAACACAGATCG |
| | | − CGATCTGTGTTTGTCTGA |
| | Reference: | Tissue: colon_133990_18461 |
| | | Cancer Temp: 52° C. Primer Temp: 52° C. |
| 50 | Cancer Oligo: | + GAGAATACTGATTGAGACCTA |
| | | − TAGGTCTCAATCAGTATTCTC |
| | Reference: | Tissue: colon 134014 18566 |
| | | Cancer Temp: 58° C. |
| 51 | Cancer Oligo: | + CCAGCCAGCACCCAGGC |
| | | − GCCTGGGTGCTGGCTGG |
| | Primer Oligo: | + CCAGCCAGCACCCAGG |
| | | − CCTGGGTGCTGGCTGG |
| | Reference: | Tissue: colon_78026_722 |
| | | Cancer Temp: 60° C. Primer Temp: 56° C. |
| 52 | Cancer Oligo: | + TAGACCAACAGAGTTCC |
| | | − GGAACTCTGTTGGTCTA |
| | Reference: | Tissue: colon_121771_670 |
| | | Cancer Temp: 50° C. |
| 53 | Cancer Oligo: | + CTAGGTACGAGGCTGGGTTTT |
| | | − AAAACCCAGCCTCGTACCTAG |
| | Primer Oligo: | + CTAGGTACGAGGCTGGG |
| | | − CCCAGCCTCGTACCTAG |
| | Reference: | Tissue: colon_121801_753 |
| | | Cancer Temp: 64° C. Primer Temp: 56° C. |
| 54 | Cancer Oligo: | + CGAGGCGGGTGTTAATCGGCC |
| | | − GGCCGATTAACACCCGCCTCG |
| | Primer Oligo: | + CGAGGCGGGTGTTAATC |
| | | − GATTAACACCCGCCTCG |
| | Reference: | Tissue: colon_123056_2392 |
| | | Cancer Temp: 70° C. Primer Temp: 54° C. |
| 55 | Cancer Oligo: | + AAGGCTAGGTAGAGGCTG |
| | | − CAGCCTCTACCTAGCCTT |
| | Reference: | Tissue: colon_123353_2625 |
| | | Cancer Temp: 56° C. |
| 56 | Cancer Oligo: | + CATGGCCATGCTGTGCA |
| | | − TGCACAGCATGGCCATG |
| | Reference: | Tissue: colon_123371_2693 |
| | | Cancer Temp: 54° C. |
| 57 | Cancer Oligo: | + AGGTACGAGGCCGGTGTTAATCGGCCGA |
| | | − TCGGCCGATTAACACCGGCCTCGTACCT |
| | Primer Oligo: | + AGGTACGAGGCCGGTG |
| | | − CACCGGCCTCGTACCT |
| | Reference: | Tissue: colon_123372_2695 |
| | | Cancer Temp: 90° C. Primer Temp: 54° C. |
| 58 | Cancer Oligo: | + TGCACCACAAGCAAACAGGCC |
| | | − GGCCTGTTTGCTTGTGGTGCA |
| | Primer Oligo: | + TGCACCACAAGCAAACAG |
| | | − CTGTTTGCTTGTGGTGCA |
| | Reference: | Tissue: colon_123799_3379 |
| | | Cancer Temp: 66° C. Primer Temp: 54° C. |
| 59 | Cancer Oligo: | + TGCTGCCCTCAATGGTC |
| | | − GACCATTGAGGGCAGCA |
| | Reference: | Tissue: colon_124226_4533 |
| | | Cancer Temp: 54° C. |
| 60 | Cancer Oligo: | + AGGCCGGTGGTTAATCGGCCGAGG |
| | | − CCTCGGCCGATTAACCACCGGCCT |
| | Primer Oligo: | + AGGCCGGTGGTTAATCG |
| | | − CGATTAACCACCGGCCT |
| | Reference: | Tissue: colon_124431_5222 |
| | | Cancer Temp: 80° C. Primer Temp: 54° C. |
| 61 | Cancer Oligo: | + GAGGCCGGTGGTTAATCGGCCGAG |
| | | − CTCGGCCGATTAACCACCGGCCTC |
| | Primer Oligo: | + GAGGCCGGTGGTTAATC |
| | | − GATTAACCACCGGCCTC |
| | Reference: | Tissue: colon_124442_5305 |
| | | Cancer Temp: 80° C. Primer Temp: 54° C. |
| 62 | Cancer Oligo: | + GCTAGGTACGAGGCTGGGTTTT |
| | | − AAAACCCAGCCTCGTACCTAGC |
| | Primer Oligo: | + GCTAGGTACGAGGCTGG |
| | | − CCAGCCTCGTACCTAGC |
| | Reference: | Tissue: colon_124449_5356 |
| | | Cancer Temp: 68° C. Primer Temp: 56° C. |
| 63 | Cancer Oligo: | + AACATACGGCTAGGTACGA |
| | | − TCGTACCTAGCCGTATGTT |
| | Reference: | Tissue: colon_124461_5420 |
| | | Cancer Temp: 56° C. |
| 64 | Cancer Oligo: | + GGTGGTAATCGGACGAGG |
| | | − CCTCGTCCGATTACCACC |
| | Reference: | Tissue: colon_124495_5584 |
| | | Cancer Temp: 58° C. |
| 65 | Cancer Oligo: | + GGGTGATCGGACGAGGC |
| | | − GCCTCGTCCGATCACCC |
| | Reference: | Tissue: colon_124565_5924 |
| | | Cancer Temp: 58° C. |
| 66 | Cancer Oligo: | + ACATGCCTAGGGTTCAA |
| | | − TTGAACCCTAGGCATGT |
| | Reference: | Tissue: colon_128283_2235 |
| | | Cancer Temp: 50 |

These 59 cancer markers include many SNPs, but they also include longer mutations.

Cancer marker supersets specific for other types of cancers have also been identified. Cancer markers for lung cancer are provided in TABLE 3 and those for lymph cancer in TABLE 4.

TABLE 3

Lung Cancer Marker Subset
(SEQ ID NOS: 345-368)

| | |
|---|---|
| A Cancer Oligo: | + TGAGACAGCTCATCACA |
| | − TGTGATGAGCTGTCTCA |
| Reference: | Tissue: lung_97380_1525 |
| | Cancer Temp: 50 C. |
| | Primer Temp: 50 C. |
| B Cancer Oligo: | + TCTGGACTGATCTAACA |
| | − TGTTAGATCAGTCCAGA |
| Reference: | Tissue: lung_114200_11255 |
| | Cancer Temp: 48 C. |
| | Primer Temp: 48 C. |
| C Cancer Oligo: | + CAAGTTCCTATAGGAGT |
| | − ACTCCTATAGGAACTTG |
| Reference: | Tissue: lung_116399_15887 |
| | Cancer Temp: 48 C. |
| | Primer Temp: 48 C. |
| D Cancer Oligo: | + TGCCATAAACTGGGTTA |
| | − TAACCCAGTTTATGGCA |
| Reference: | Tissue: lung_107413_2916 |
| | Cancer Temp: 48 C. |
| | Primer Temp: 48 C. |
| E Cancer Oligo: | + GGCTAGGTACGAGGCTGGGTGTG |
| | − CACACCCAGCCTCGTACCTAGCC |
| Primer Oligo: | + GGCTAGGTACGAGGCTG |
| | − CAGCCTCGTACCTAGCC |
| Reference: | Tissue: lung_99814_4327 |
| | Cancer Temp: 76 C. |
| | Primer Temp: 56 C. |
| F Cancer Oligo: | + AAACCTGCAATATGATG |
| | − CATCATATTGCAGGTTT |
| Reference: | Tissue: lung_124202_2868 |
| | Cancer Temp: 46 C. |
| | Primer Temp: 46 C. |
| G Cancer Oligo: | + GCGTGATGGCGGGGGGCTCT |
| | − AGAGCCCCCCGCCATCACGC |
| Primer Oligo: | + GCGTGATGGCGGGGG |
| | − CCCCCGCCATCACGC |
| Reference: | Tissue: lung_98869_3329 |
| | Cancer Temp: 70 C. |
| | Primer Temp: 54 C. |
| H Cancer Oligo: | + GCTTACATCCGTGATGT |
| | − ACATCACGGATGTAAGC |
| Reference: | Tissue: lung_108655_5362 |
| | Cancer Temp: 50 C. |
| | Primer Temp: 50 C. |
| I Cancer Oligo: | + TTACTCTCATGTGGCCAA |
| | − TTGGCCACATGAGAGTAA |
| Reference: | Tissue: lung_123536_1762 |
| | Cancer Temp: 52 C. |
| | Primer Temp: 52 C. |
| J Cancer Oligo: | + TCTGATGAACAGAAGAAG |
| | − CTTCTTCTGTTCATCAGA |
| Reference: | Tissue: lung_125101_4407 |
| | Cancer Temp: 50 C. |
| | Primer Temp: 50 C. |

TABLE 4

Lymph Cancer Marker Subset
(SEQ ID NOS: 369-398)

| | |
|---|---|
| i Cancer Oligo: | + GCTGAACCTGCGACTGGTA |
| | − TACCAGTCGCAGGTTCAGC |
| Primer Oligo: | + GCTGAACCTGCGACTGG |
| | − CCAGTCGCAGGTTCAGC |
| Reference: | Tissue: lymph_67664_6573 |
| | Cancer Temp: 60 C. |
| | Primer Temp: 56 C. |
| ii Cancer Oligo: | + TAGGTACGAGGCTGGGT |
| | − ACCCAGCCTCGTACCTA |
| Reference: | Tissue: lymph_55415_7578 |
| | Cancer Temp: 54 C. |
| | Primer Temp: 54 C. |
| iii Cancer Oligo: | + GGCTAGTACGAGGCTGGGT |
| | − ACCCAGCCTCGTACTAGCC |
| Primer Oligo: | + GGCTAGTACGAGGCTGG |
| | − CCAGCCTCGTACTAGCC |
| Reference: | Tissue: lymph_55600_7985 |
| | Cancer Temp: 62 C. |
| | Primer Temp: 56 C. |
| iv Cancer Oligo: | + CTAGGTACGAGGCTGGGTG |
| | − CACCCAGCCTCGTACCTAG |
| Primer Oligo: | + CTAGGTACGAGGCTGGG |
| | − CCCAGCCTCGTACCTAG |
| Reference: | Tissue: lymph_60248_7359 |
| | Cancer Temp: 62 C. |
| | Primer Temp: 56 C. |
| v Cancer Oligo: | + GTACGAGGCTGGGTGTT |
| | − AACACCCAGCCTCGTAC |
| Reference: | Tissue: lymph_60270_7430 |
| | Cancer Temp: 54 C. |
| | Primer Temp: 54 C. |
| vi Cancer Oligo: | + GAAACTGTTGGCGTGAT |
| | − ATCACGCCAACAGTTTC |
| Reference: | Tissue: lymph_50077_1076 |
| | Cancer Temp: 50 C. |
| | Primer Temp: 50 C. |
| vii Cancer Oligo: | + GAGCAGAAACGGGAGACCTG |
| | − CAGGTCTCCCGTTTCTGCTC |
| Primer Oligo: | + GAGCAGAAACGGGAGAC |
| | − GTCTCCCGTTTCTGCTC |
| Reference: | Tissue: lymph_69924_10602 |
| | Cancer Temp: 64 C. |
| | Primer Temp: 54 C. |
| viii Cancer Oligo: | + GGCCTTCGAGCGGGGTGTTGGGG |
| | − CCCCAACACCCCGCTCGAAGGCC |
| Primer Oligo: | + GGCCTTCGAGCGGGG |
| | − CCCCGCTCGAAGGCC |
| Reference: | Tissue: lymph_50152_1336 |
| | Cancer Temp: 80 C. |
| | Primer Temp: 54 C. |
| ix Cancer Oligo: | + AGTTTCTTCAAGATCAC |
| | − GTGATCTTGAAGAAACT |
| Reference: | Tissue: lymph_62939_1828 |
| | Cancer Temp: 46 C. |
| | Primer Temp: 46 C. |
| x Cancer Oligo: | + GAGGAAGTAATCTGCCC |
| | − GGGCAGATTACTTCCTC |
| Reference: | Tissue: lymph_13680_599 |
| | Cancer Temp: 52 C. |
| | Primer Temp: 52 C. |

Samples Tested

The cancer detection reagents discussed herein may be used on any sample likely to contain the cancer markers. However, in preferred embodiments, the markers are detected in an easily obtainable bodily fluid, such as peripheral blood. Use of peripheral blood may also provide the advantage of allowing markers from several differentiated tumors in the same subject to be detected at once. Yet there may be circumstances, such as when information about only one tumor is desired, in which tissue samples or other samples are examined.

Cancer tissue samples and biopsies usually come from a single tumor, even when multiple tumors are present. In the early stages of cancer most cancer cells are daughters of a parent tumor and often have the same mutations as the cells in the tumor. However, metastatic cancer cells often have different mutations. Further, metastatic tumors, even if initially similar, follow different development pathways and may accumulate different additional mutations over time. Finally, it is well known that many cancer treatments cause further mutations in cancer cells. Therefore, cancer cells in later stages of cancer often do not have the same mutations as those in early stages. Variation in mutations is also often seen among metastatic tumors in the same individual.

Because tumors tend to have individual mutations, it follows that a tissue sample taken from a single tumor will likely not contain all the cancer mutations found throughout a subject's cancer. A profile of all or most mutations in the subject's body using traditional methodologies would thus require samples from multiple tumors. In contrast, in embodiments of the present invention using blood as a sample, all or most of the mutations present in metastatic cancer may be detected in a single sample because it contains cells from multiple tumors. Further a blood sample may even contain cells from small metastatic tumors not detectable using conventional techniques.

Diagnostic Uses

The cancer markers of the present invention and corresponding cancer detection reagents may be used in diagnosis of metastatic cancer, particularly pathology-based diagnosis, including initial diagnosis as well as treatment and disease progression monitoring, and also including monitoring of targeted cancer cell death.

In a preferred embodiment, the present invention is used to detect a plurality of cancer markers to provide a cancer marker profile of the subject. The markers tested may be selected based on a variety of factors. Two factors include overall likelihood of occurrence in any type of cancer, or association with a cancer originating in a particular tissue.

The screening methods of the present invention may be used for a variety of diagnostic purposes. For purposes of this specification, “diagnostic” refers not only to initial determinations of whether a subject has a disease, but also to any test to examine the nature of a disease. For example, forms of diagnosis in the present specification may include screening in a healthy subject or a subject with symptoms to initially determine whether cancer is present, testing at any point after a subject has been determined to have cancer, testing to help recommend or monitor a course of treatment, prognostic testing, testing to monitor the development of cancer, including the development of any new mutations, and testing to determine the presence or absence or eradication of metastatic cells.

For example, the methods of the present invention may be used to detect the presence of cancer cells, particularly metastatic cancer cells or other cancer cells found in the blood. The methods may be used for initial diagnosis of cancer or metastatic cancer, even when tumors are too small to be detected by imaging or other techniques.

Screening according to the present invention may be used to not only indicate the presence of cancer cells, but also to determine some or all of the mutations or abnormalities present in these cells. Knowledge of the mutations present may be used in directing treatment. For example, drugs known to be effective against certain types of cancer only may be prescribed or avoided based on the underlying mutations of a subject's cancer. Additionally, knowledge of subject-specific cancer mutations may be used to develop new classes of cancer drugs, including subject-specific cancer drugs targeted to the diagnosed mutations. These targeted drugs may affect the mutant proteins, particularly cell-surface proteins, or they may act on cellular nucleic acids, such as mRNA.

Further, additional testing incorporating regions flanking the cancer marker sites may be used to determine the specific genes affected by a cancer marker in a given cancer patient. As TABLEs 1 and 2 clearly show, while some cancer markers are associated with only a few genes, most have been found in a number of genes. The function of some of these genes is known. Accordingly, the ability to determine in which gene a cancer marker lies provides additional information that may be used to direct cancer treatment.

Given the way public data is generated, one would expect much chance and coincidence in any commonality or lack thereof between the cancer markers and cancer cell lines. However, FIG. 5 suggests that some cancer markers appear in some cell lines while others appear in different cell lines. This suggests that some cancer markers are found in some cancer subjects while others are found in different cancer subjects. Each cancer subject is expected have mRNA containing a subset of cancer markers constituting an individual cancer profile, and identifying which genes may be mutated in that individual. It is possible however, that with a large enough subject pool, the same cancer profile may be observed among different subjects, but nevertheless one does not expect every subject in the pool to have an identical cancer profile.

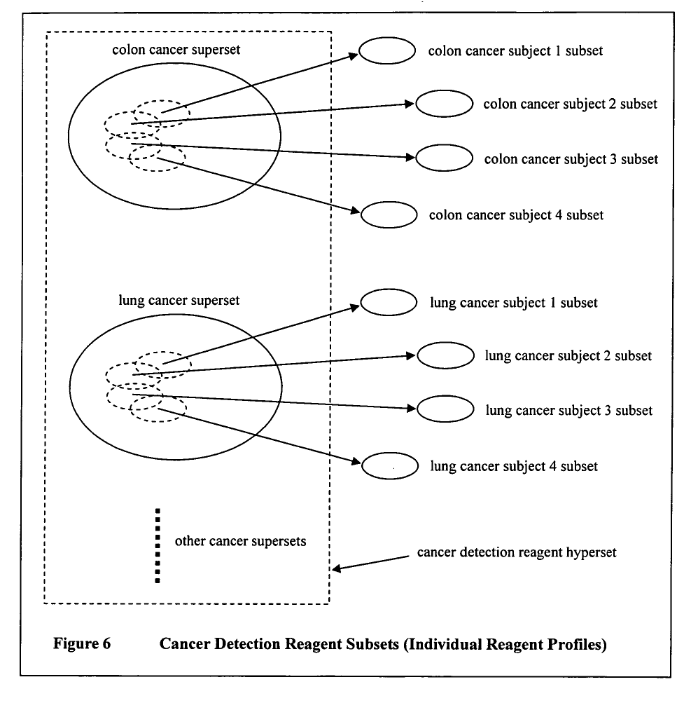

The extent of individualism in cancer is not clearly understood. However, individuality nevertheless appears to correlate with cancer type, as illustrated in FIG. 6. The cancer marker hyperset may constitute all mRNA molecules of length 17-mer or greater that are exclusive to cancer cells. Each cancer type then has a corresponding cancer marker superset, and each cancer subject has a cancer marker subset, which is synonymous to their individual cancer profile.

Because TABLEs 1 and 2 present a set of cancer markers found in a variety of different cancer cells, one should not expect to find all of them in a single cancer subject, although this is not impossible. Rather, the 59 cancer markers of TABLEs 1 and 2 or subcombinations thereof are useful in generating a cancer profile for a particular subject's cancer. By including a large number of cancer markers in any assay or set of assays, a more complete cancer profile may be developed. Additionally, knowledge of what cancer markers are not present in subject's mRNA may also be very useful for diagnosis, including prognosis, as well as cancer progression and treatment monitoring. It may, for example, be useful in selecting a treatment for the subject.

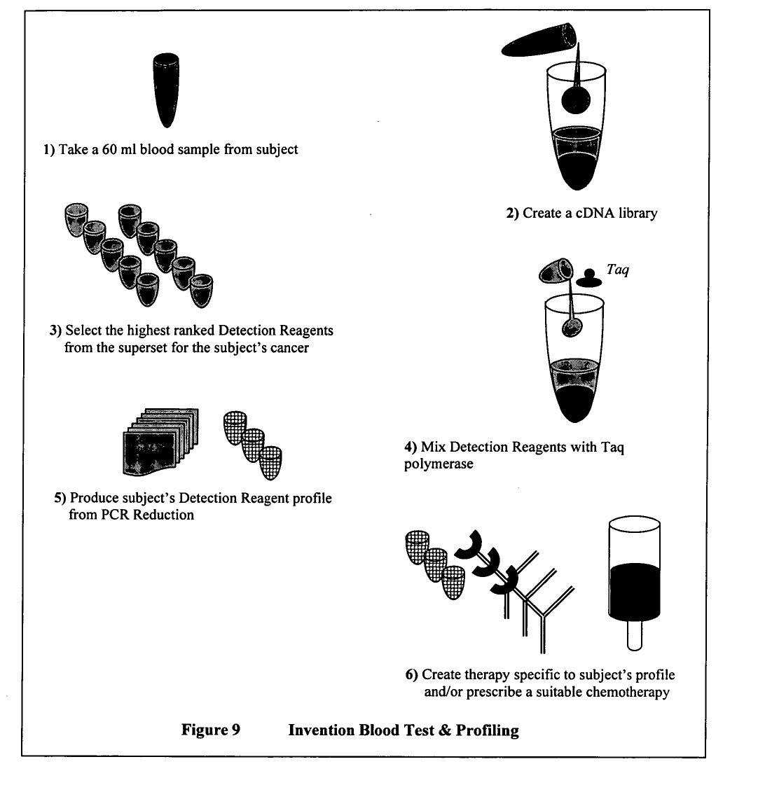

Cancer profiles may be created for cancer subjects using a blood sample and the methodologies described herein. FIG. 9 illustrates steps for one such exemplary methodology. In most instances, a cancer profile may be obtained within a few hours to a few days after obtaining a blood sample from a subject.

Because most cancer markers are associated with a group of genes, one may quickly determine which group of genes are mutating in a subject's cancer in a way that is exclusive to cancer cells. Any subsequent therapy can utilize this genetic information for specific cancer cell targeting. Unfortunately, most existing therapies do not have this kind of targeting capacity. Therefore, the blood-based tests of the present invention may also be precursory tests for new therapeutics that can use the cancer detection reagents for specific cancer cell targeting.

In a specific embodiment of the present invention, three general types of assays are provided. The first type of assay examines a sample for the presence or absence of cancer markers common in multiple types of cancers. In a preferred embodiment, the testing subset of cancer markers is selected based on their frequency of occurrence in cancers represented in the general cancer hyperset. For example, all cancer markers that have been found in more than a certain number of cancers may be selected. Alternatively, the cancer markers may be ranked in frequency of occurrence and a certain number of them may be selected. For example, the top 300 cancer markers may be selected for use in the diagnostic assay.

As new cancer samples are added to the hyperset, this has had little significant effect on the relative frequencies with which cancer markers are found in cancer tissue. This indicates that the hyperset is representative of cancer overall and that there are some cancer markers that are simply far more likely to appear in any type of cancer than others.

A general diagnostic assay that examines cancer markers from the general cancer marker hyperset might be used, for example, as part of routine screening, such as yearly blood tests. It might also be used for individual with symptoms, such as weight loss, consistent with both cancer and many other diseases.

A second type of assay may focus on a particular type of cancer, such as colon cancer. Like the general assay, this assay might look for a subset of cancer markers occurring at above a certain frequency, or it might look for a certain number of top markers in a frequency ranked list. Cancer marker supersets for specific cancers also exhibit little change in the relative frequency of higher frequency markers as new data is added.

This second type of assay might be used for a subject known to have a specific type of cancer. It might provide a more detailed indication of the mutations present in that subject's cancer than can be obtained using a general cancer assay. It might also provide a more detailed prognosis or treatment plan.

The third type of assay determine which genes are affected by a subject's cancer mutations. This assay may be used at any point, but for cost and efficiency reasons, may be focused on specific cancer markers, and may be used only for subjects previously shown to have those cancer markers. However, in some embodiments, such as those focusing on common cancer markers, it may be efficient to screen for affected genes concurrently with the cancer marker screen.

This third type of assay may detect specific genes by also examining the flanking nucleic regions around the cancer marker. These flanking regions tend to differ from gene to gene. Flanking regions suitable for a given assay method and able to distinguish potentially affected genes from one another will be apparent to one skilled in the art.

Cancer Marker Profiles

Cancer marker profiles may be developed for individual subjects. These subjects are most often a human, such as a human having or suspected of having cancer. However, subjects may also include other mammals. Subjects may include patients. In certain contexts, the subject may be a tumor or suspected tumor.

Cancer marker profiles include the identity of a cancer marker and an indication of whether it was detected in the subject. Cancer marker profiles generally provide this information for more than one cancer marker. Cancer marker profiles may provide results in a simple positive/negative format. They may also indicate an amount of cancer marker found either quantitatively or qualitatively. Finally, cancer marker profiles may include information about the gene or genes in which a cancer marker is found in a subject.

All mammals accumulate somatic mutations as they age. Experiments have shown that healthy tissue is free of cancer markers. However, because blood often contains aberrant cells found anywhere in the body, it is likely that an adult mammal, or even a juvenile, will exhibit some cancer markers in its blood.

The presence of some cancer markers in a subject's blood does not necessarily indicate that the subject has cancer. Rather, the number, type, or combination of cancer markers is likely indicative of whether the subject has cancer. For any given set of cancer markers, routine experimentation comparing blood from healthy individuals with that from patients known to have cancer should readily reveal which cancer marker profiles are indicative of cancer and which are not. Further, long-term studies that track whether healthy subjects develop cancer, when, and what their cancer marker profiles were over the course of the study should reveal cancer marker profiles that are indicative of an increased propensity to develop cancer This information may be used to guide preventative measures or early cancer treatment.

Diagnosis Protocols and Examples

Cancer markers in a sample may be identified using any appropriate method. However, in a specific embodiment, cancer markers may be identified by PCR analysis of a peripheral blood sample. PCR analysis may include RT-PCR, in which mRNA from the sample is converted to cDNA. This cDNA is then subject to PCR Reduction. Further, PCR analysis may be very readily tailored to include detection of flanking regions, allowing analysis of which gene is affected by a cancer marker.

PCR Reduction

Traditional PCR amplifies a set region of nucleic acid located between the 5′ and 3′ primers. Because both 5′ and 3′ primers are used, the newly created nucleic acid strand becomes available as a template in the next cycle. All primers and PCR conditions are not equally effective at amplification, thus some create new templates at a higher rate than other primers. The effect combined with the ability of new strands to serve as templates results in significant differences in the number of individual nucleic acid strands having the amplified sequence when different primers are used. This difference is related to primer and PCR-condition efficiency rather than the actual number of template strands that were available in the original sample.

A more accurate comparison of the numbers of mRNA molecules containing different cancer markers in a given sample may be obtained using a modified type of PCR herein referred to as “PCR Reduction”. Using this methodology, only 5′ primers are provided. These primers are able to hybridize with the original template nucleic acid, but not with any strands produced as part of the PCR process because such strands contain sequences identical to, but not complementary to the 5′ primer. Because only the original template nucleic acid may serve as a template for the PCR reaction, differences in copy number of different cancer detection reagent sequences due to primer or PCR efficiency are not so pronounced. Copy number has a much closer correlation with actual number of original templates.

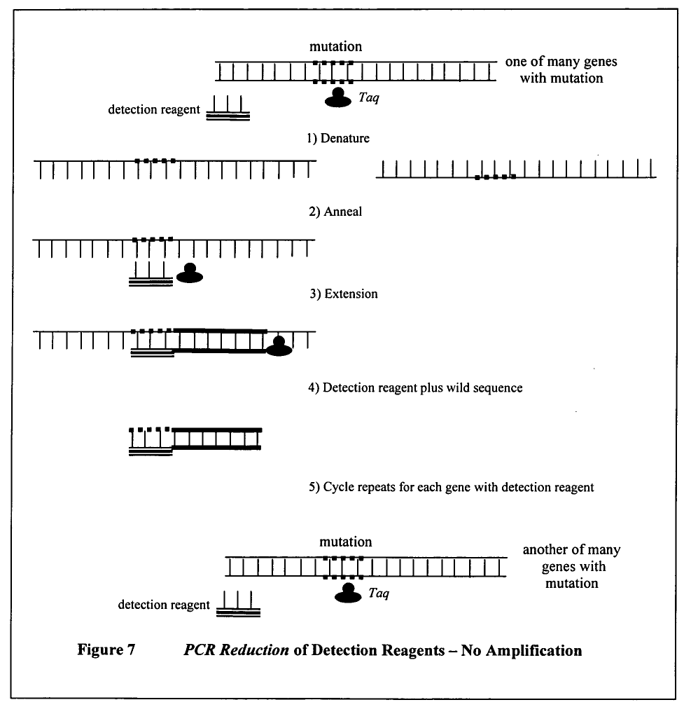

In PCR Reduction, polymerization occurs until the polymerase falls off of the template strand. This tends to leave a trailing end after the 5′ primer. These trailing ends vary somewhat in length, but normally all terminate within several hundred base pairs of the primer. Thus, all of the PCR reaction products may be resolved via electrophoresis on a gel as a single, but slightly blurry band. One example PCR Reduction methodology is illustrated in FIG. 7.

Although amplification of the cancer markers alone might be useful in some embodiments of the invention, in the PCR Reduction technique described above the tailing end allows for easy gel-based detection that could not be easily achieved using the small cancer detection reagents alone. If there is no cancer detection reagent sequence present in the sample, then the primers have no template and no band shows up at the expected location after electrophoresis. On the other hand, if the cancer detection reagent sequences are present, a blurry band is present. The intensity of this band may be analyzed using conventional techniques to estimate the relative abundance of templates in the sample containing each detection reagent sequence.

Although it is difficult to detect which gene contains the particular cancer marker using PCR Reduction and a gel alone, such information can be determined through further analysis of the PCR Reduction product. For example, traditional PCR using primers specific to different genes may be performed on the PCR Reduction product. Because the PCR Reduction primer correlates with the cancer marker, but transcription occurs for up to several hundred base pairs, the trailing end will normally be of sufficient length to allow different genes to be distinguished. It is also possible to sequence the PCR Reduction products to determine which gene or genes contain the cancer marker.

MicroArrays

In another embodiment, a microarray may be constructed based on cancer markers. Cancer detection reagents including these markers may be placed on the microarray. These cancer detection reagents may be different than those used in PCR methods. However, they should be designed and used in conditions such that only nucleic acids having the cancer marker may hybridize and give a positive result. Microarray-based assays are also very amenable to detection of flanking regions, allowing identification of specific affected genes.

Most existing microarrays, such as those provided by Affymetrix (California), may be used with the present invention. Microarrrays specifically able to detect SNPs or small deletions may be particularly useful, as many cancer markers fall in these two categories of abnormalities.

In particular embodiments, three types of microarrays may be provided that roughly correspond to the three types of assays described above. Specifically, a general cancer marker microarray may be provided, for example for use in general screening. Another type of microarray, each for a specific type of cancer, may be provided, for example for more detailed diagnosis of a subject known to strongly suspected to have a given type of cancer. Finally, a third type of microarray able to distinguish the gene affected by a cancer marker may be provided. This type of microarray may be tailored to one cancer marker, or it may be able to detect specific affected genes for a number of cancer markers.

Hybrid microarrays able to do multiple types of assays on the same array are also possible. For example, a single microarray may be able to both detect cancer markers and determine the affected genes for those markers.

Other Assays

In additional embodiments, other methods of nucleic acid analysis may be used. For example, FACS bead-based assays, such as those available for nucleic acid analysis through Luminex (Texas) or Becton-Dickinson (New Jersey) may be used to detect cancer markers and gene-identifying flanking sequences.

Finally, peptide-based assays are also possible. Because the cancer markers were identified through mRNA analysis, it is expected that most of them will be expressed as an aberrant protein. These assays may be particularly useful for cancer markers often found in surface proteins, although cells may be readily lysed to allow access to internal proteins as well. Peptide analysis using antibodies may be particularly useful, as such antibodies may have later applications in treatment.

Kits and Services

The cancer markers of the present invention may be detected using kits. These kits may include cancer detection reagents suitable for a particular type of assay. Other reagents useful in the assay may be included in the kit. Use of the kit may result in a cancer marker profile for the subject. Kits may be designed for use in any aspect of medical testing, including laboratory research, commercial diagnostic laboratory testing, hospital or clinic laboratory testing, or physician's office testing. Kits may require specific additional equipment, such as a PCR cycler, microarray reader, or FACS machine.

The present invention may also be supplied commercially as a testing service. For example, a sample may be provided to a commercial testing laboratory which then uses appropriate cancer detection reagents and assay to determine the cancer profile for the sample. The results may then be returned to the entity providing the sample.

Uses of Diagnostic Results

Diagnostic results may be used to direct the treatment of a patient who appears to have cancer or to be likely to develop cancer in a number of manners. The patient may be given preventative treatment based on the presence of a large number of cancer markers or certain combinations. The patient may also be treated differently depending on the stage of the disease. Treatment may be varied as the disease and cancer markers change.

Treatment itself may include conventional treatments, such as chemotherapy. It may also include antibody or antisense therapy based on the particular cancer profile of the patient. The patient's cancer markers may be used to develop antibodies to a cancer marker specific epitope. They may also be used to develop antisense molecules that will interfere with the cellular mechanisms of cancer cells, but not normal cells.

Because the cancer detection reagents of the present invention are absent in the healthy cell transcriptome, they represent cancer-specific targets for inducing cancer cell death. For example, although some cancer detection reagents may be translated into peptides located primarily within the cell, some are embedded in sequences that normally encode extracellular or membrane proteins. Such sequences are readily known to the art and are considered predictive of the likely cellular location of a protein and portions of it. Accordingly, particularly for proteins with extracellular regions, administration of an antibody specific for a peptide encoded by a cancer detection reagent is expected to induce cell death. Because only cancer cells exhibit these peptides, only cancer cells are targeted and killed by the antibodies.

Antibodies used in conjunction with the present invention may include monoclonal and polyclonal antibodies, non-human, human, and humanized antibodies and any functional fragments thereof.

Although a single cancer detection reagent may be used to target multiple genes or gene products in the methods of inducing cancer cell death of the present invention, in some embodiments multiple cancer detection reagents may be targeted to produce an potent effect. Combined agents targeting more than one cancer detection reagent may also be particularly useful if administered to a subject with multiple tumors. The subject's tumors may have differentiated such that every tumor does not contain any one cancer detection reagent sequence. Incorporating agents targeted to multiple cancer detection reagent sequences may allow these differentiated cancer cells to be killed more effectively. Such combined approaches are particularly powerful against new or small tumors that may not be detected using conventional methods, but nevertheless contain a cancer detection reagent sequence detectable when diagnostic methods of the present invention are used to create a cancer profile.

Thus, targeted cancer cell death may be accomplished according to selected methods of the present invention according to a three-step method. First, a cancer profile may be created for the subject. Second, a targeted cancer cell death agent may be created and tested on the subject's blood or other tissue sample. Third, the agent may be administered to the subject to cause targeted death of cancer cells in that subject. This process may be accomplished in as little as three weeks.

Continued monitoring may allow detection of the disappearance of any cancer detection reagents in the subject as well as the appearance of any new ones. The agent or combination of agents administered to the subject may then be changed accordingly.

### EXAMPLES

The following examples are included to demonstrate specific embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

### Methods, Reagents and Subject Background

To accomplish these tests, two volunteers with phase 4 metastasized colon cancer were selected. These volunteers are herein referred to as subject R and subject H. Subject R is a female. Subject R provided a 9 mm, excised tumor for testing as well as a 60 mL peripheral blood sample. Subject H is a male. Subject H provided a 60 mL peripheral blood sample.

cDNA libraries were constructed from all samples. A cDNA library was also constructed from a pool of random tissue samples from healthy, cancer-free individuals. This cDNA pool represents the normal, non-cancerous sample in these Examples.

### Example 2

### Cancer Marker Sets

By comparing mRNA from cancer cells, as reported in public databases, with normal human mRNA, also as reported in cancer databases, using a proprietary computer program, a number of cancer markers have been identified. These cancer markers have been frequency ranked. Because generally each sample of cancer cells used for reporting in the public database was obtained from a different patient, each occurrence of a cancer marker in the databases correlates with an occurrence in an actual human subject. Thus, the frequency of occurrence in the databases roughly corresponds with the past and expected future frequency at which a cancer marker appears.

Cancer markers have been ranked based on frequency for each type of cancer examined. Additionally, the present invention reveals that many cancer markers are often found in multiple types of cancer. Thus, markers have been ranked based on their frequency of occurrence overall in all cancer examined.

Some colon cancer markers identified thus far that are also good general cancer markers are provided in TABLEs 1 and 2.

### Example 3

### Blood Sample Preparation

60 mL of peripheral blood was collected using a standard IV phlebotomy needle in purple top a vacuum tube containing EDTA. Tubes containing heparin may also be suitable. The blood was then stored at 4° C. until further processing. Processing was completed as quickly as possible in order to lessen RNA degradation.

Total RNA was isolated using a QIAamp RNA blood mini kit. (Quiagen, California) The total yield of RNA was approximately 60 μg.

Later tests revealed that blood samples were prepared using Trizol reagent (Invitrogen, California) yielded approximately 400 μg. However, these samples were not used in the present examples.

Blood may also be collected in tubes containing pre-aliquoted stabilization reagents, such as Paxgene Blood RNA tubes (Quiagen, California). Paxgene tubes hold 2.5 ml/blood per tube and the blood normally remains stable at room temperature for 5 days. Paxgene tubes are specifically designed to prevent RNA degradation as well as gene induction that sometimes occurs after blood is collected.

### Example 4

### Primers for PCR Testing

Typical primer data as provided by the manufacturer is as follows.
Synthesis scale: 200 nmol
Length: 17-mer
Molecular Weight (Ammonium Salt): 5383.4
Exact Weight per OD (Ammonium Salt): 32.34
Nanomoles per OD (Ammonnium Salt): 6.12
Millimolar Extinction Coefficient: 163.35
Total ODs in This Tube: 20
Total Amount in μg: 646.76
Total Amount in nmoles: 122.44
Purification: Desalted
Melting Temperature (Celsius): 56.0
5′ End: OH
3′ End: OH

For each of the 59 general cancer markers identified in TABLE 2, PCR Primers for the markers identified as well as PCR conditions are provided.

### Example 5

### cDNA Synthesis

Prior to cDNA synthesis, residual DNA was removed from the total RNA by DNAase I digestion. Specifically, a reaction mixture was created having a total volume of 10 μL and containing 5 μg of total RNA, 1 μL of 10× buffer and 1 μL of DNAase I. This mixture was maintained at room temperature for 15 minutes, then 1 μL of 25 mM EDTA was added. The EDTA mixture was incubated for 15 minutes at 65° C., then placed on ice for 1 minute. The reaction was collected by centrifugation.

A SuperScript III kit (Invitrogen, CA) was used for first strand cDNA synthesis from the DNAase I digested total RNA samples. A poly T primer was used. However, a random primer may also be used. Random primers may be particularly desirable if the cancer marker is located far upstream of the polyT tail of an mRNA.

Approximately 10 μL of DNAase I digested RNA was mixed with 1 μL of 10 mM dNTP and 1 μL of oligodT (0.5 μg/pL) primer. This RNA/primer mixture was incubated at 65° C. for 5 minutes, then placed on ice for 1 minute.

A reaction mixture was prepared containing 2 μL of 10×RT buffer, 4 μL of 25 mM MgCl₂, 2 μL of 0.1 M DTT, and 1 μL of RNAase Out (Invitrogen, California). 9 μL of reaction mixture was added to the RNA/primer mixture. The total mixture was collected by centrifugation then incubated at 42° C. for 2 minutes. 1 μL (50 units) of Superscript III RT (Invitrogen, California) was then added and the resulting mixture was incubated at 42° C. for 50 minutes. The reaction was terminated by incubation at 70° C. for 15 minutes or at 85° C. for 5 minutes, followed by chilling on ice. The reaction was collected by centrifugation.

Finally, 1 μL of RNAase H was added and the sample was incubated for 20 minutes at 37° C. to degrade the remaining RNA.

Each sample was treated in this manner. The single cDNA sample created was then used as the starting material for each subsequent PCR Reduction reaction.

### Example 7

### PCR Reduction

PCR Reduction was used to amplify any cancer markers in the cDNA. As explained above, PCR reduction gives a more accurate picture of relative amounts of mRNA carrying a cancer marker in the sample because it does not result in products that can themselves become templates for amplification. Rather, through use of only one primer, only the original templates are available for amplification throughout the reaction.

A PCR reaction mixture was created having a total volume of 20 μL and containing 13.8 of μL DEPC-treated water, 2 μL of 10×PCR buffer without Mg, 1 μL of 25 mM MgCl₂, 0.5 μL of 10 mM dNTP mixture, 1 μL of 20 μM antisense primer (cancer detection reagent), 1.5 μL of cDNA sample, and 0.2 μL of high fidelity 5 units/μL Taq DNA polymerase.

PCR was carried out in 35 cycles. First the PCR reaction mixture was denatured at 94° C. for 5 minutes. Then, each of the 35 cycles include 30 sec of denaturation at 94° C., 30 seconds of annealing at the annealing temperature for the primer (annealing temperatures are indicated in TABLE 2), and 1 minute of extension at 72° C. Upon completion, the reactions were maintained at 4° C.

Conditions were selected to obtain amplification products in the range of 100-500 bp. Conditions may be altered to obtain different sized products.

PCR analysis of both blood and tumor tissue for two terminally ill subjects was performed using antisense cancer detection reagents corresponding to cancer markers 3 and 5-66 of TABLE 2.

### Example 8

### PCR Results

To determine whether cancer markers were present in the samples, after PCR was complete 10 μL of PCR reaction mixture was loaded on a 1% agarose gel and electrophoresis was performed. The gels were then imaged.

PCR Results are provided in TABLE 5. As the table shows, the markers identified are generally not present in normal tissue. (The one that did appear in normal tissue has been excluded from inclusion as a cancer marker, although it remains possible that it is a cancer marker that, due to gradual accumulation of somatic mutations, was present in apparently healthy tissue.)

TABLE 5 shows the results of single priming RT-PCR using the primers with the Apoptotic Sequences from TABLE 1, the three cancer samples, and a vascular wall healthy control sample. A plus sign in TABLE 5 indicates a sequence's presence and a minus sign indicates a sequence's absence. Those sequences found in the healthy control sample were discarded from the candidate Apoptotic Sequence pool, while the others are available for subsequent cell death tests.

TABLE 5
Candidate Apoptotic Sequence RT-PCR Detection Tests

| Candidate | | Human colon | Human colon | Human colon |
|---|---|---|---|---|
| Apoptotic | | cancer tumor | cancer blood | cancer blood |
| Sequence | Healthy | cDNA from | cDNA from | cDNA from |
| Number | cDNA | Subject R | Subject R | Subject H |
| 1 | + | + | + | + |
| 2 | − | − | − | − |
| 3 | − | − | − | − |
| 4 | − | − | − | − |
| 5 | − | + | + | + |
| 6 | − | − | − | − |
| 7 | − | − | − | − |
| 8 | − | + | + | + |
| 9 | − | + | + | + |
| 10 | − | − | − | − |
| 11 | − | + | + | + |
| 12 | − | + | + | − |
| 13 | − | − | + | − |
| 14 | − | + | + | + |
| 15 | − | − | − | − |
| 16 | − | + | + | + |
| 17 | − | − | + | − |
| 18 | − | − | − | |
| 19 | − | − | − | + |
| 20 | − | | + | − |
| 21 | − | − | − | + |
| 22 | − | − | + | − |
| 23 | − | + | − | − |
| 24 | − | − | − | − |
| 25 | − | − | + | + |
| 26 | − | + | + | + |
| 27 | − | − | − | − |
| 28 | − | − | + | − |
| 29 | − | − | − | − |
| 30 | − | + | + | + |
| 31 | − | + | + | − |
| 32 | − | − | + | + |
| 33 | − | + | + | + |
| 34 | − | − | − | − |
| 35 | − | + | + | + |
| 36 | − | − | − | − |
| 37 | − | − | + | + |
| 38 | − | − | − | + |
| 39 | − | − | − | − |
| 40 | − | − | − | − |
| 41 | − | − | − | − |
| 42 | − | − | + | − |
| 43 | − | − | − | − |
| 44 | − | − | − | − |
| 45 | − | − | − | − |
| 46 | − | − | − | − |
| 47 | − | − | − | − |
| 48 | − | − | + | + |
| 49 | − | − | − | − |
| 50 | − | − | − | − |
| 51 | − | − | + | + |
| 52 | − | − | − | − |
| 53 | − | − | + | + |
| 54 | − | − | − | + |
| 55 | − | + | + | + |
| 56 | − | − | + | + |
| 57 | − | + | + | + |
| 58 | + | − | + | − |
| 59 | − | − | + | + |
| 60 | − | + | + | + |
| 61 | − | − | + | + |
| 62 | − | − | + | − |
| 63 | − | − | − | + |
| 64 | − | − | + | − |
| 65 | − | − | − | + |
| 66 | − | + | + | + |

TABLE 5 also indicates that analysis of blood actually identifies more cancer markers than analysis of tumor tissue. This is true when comparing blood and tumors from different subjects and from the same subject. This likely results from the presence of multiple tumors in each subject. Different tumors have likely accumulated different mutations over time. Tumor tissue samples can only reveal the mutations in a single tumor. However, the blood analysis techniques of the present invention can reveal mutations from multiple tumors at the same time so long as their cancer markers are present in the blood.

These human sample tests have been conducted to assess: i) validity of cancer markers; ii) their individuality; and iii) the ability to select them from a superset for a random cancer subject based purely on computational ranking. The latter characteristic is significant because it is not currently practical to test the tens of thousands of cancer markers from each superset corresponding to the cancer type of the human test samples.

TABLE 1 shows both strands of cancer detection reagents used to test these samples (although only the antisense strand was actually used). Cancer markers also affect both strands of DNA in the a subject. As described above, the cancer markers were filtered through the healthy human transcriptome contained in databases and neither stand appeared. This design constraint, and the small size of the cancer detection reagents makes them optimal for in-vitro in cDNA library diagnostics. Consequently, a cancer detection reagent was created for each cancer marker in TABLE 1.

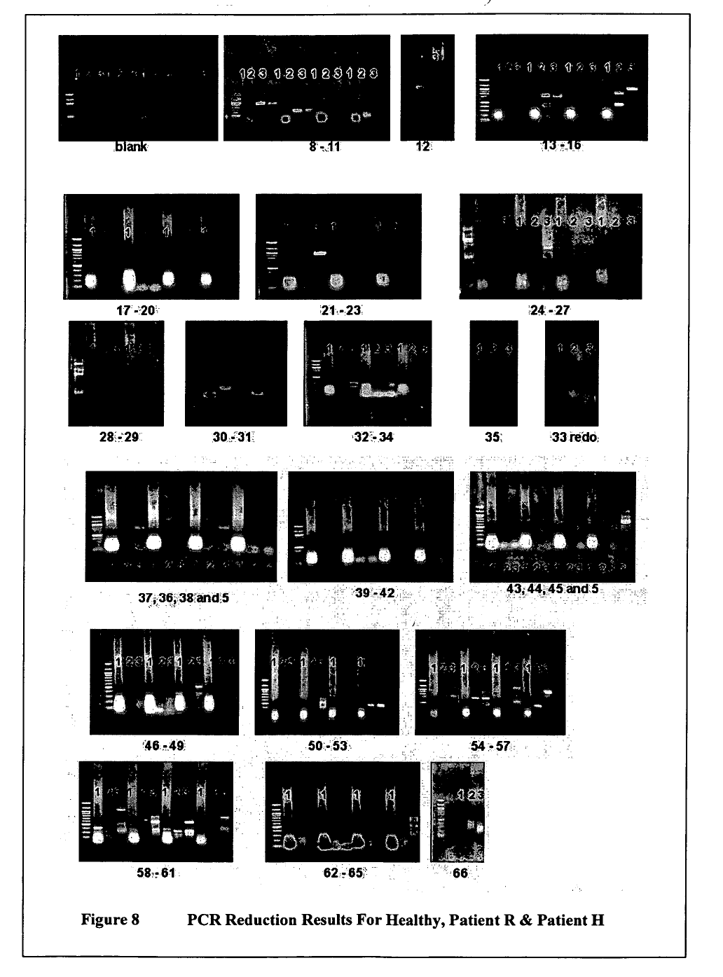

FIG. 8 shows the results from PCR Reduction using the cancer detection reagents in TABLE 1 and the cDNA from patient R's tumor, patient H's peripheral blood, and random tissue from healthy non-cancerous subjects. The healthy subject results are in lane 1, patient R results are in lane 2, and patient H results are in lane 3.

The blurred bands exhibited in FIG. 8 are caused by the variations in the trailing end lengths. Because of the absolute, cancer-if-present and healthy-if-absent nature of the cancer detection reagents, the results were interpreted as signal=cancer and no signal=healthy.

As FIG. 8 shows, the cancer detection reagents in TABLE 1 never yielded positive results from the healthy cDNA in lane 1 of the gels. (Other than cancer detection reagent 58, which, as described above, has now been excluded.)

Patient R and patient H exhibited common markers, as was expected given that both suffered from colon cancer. However, some variation was present in their cancer marker profiles as was also expected between different individuals. This reveals the individuality in the cancer marker profiles of the two subjects.

Finally, TABLE 1 includes only the highest ranked markers from the colon cancer superset. As FIG. 8 demonstrates, computational occurrences of cancer markers in specific types of cancer cell lines presents a viable ranking method for reducing the amount of in-vitro testing required to establish individual cancer marker profiles for actual human subjects.

FIG. 8 shows a varied degree of band intensity for different cancer detection reagents. Because PCR Reduction was used to in the assays, this amplitude is a good reflection of the number of mRNA transcripts containing each of the cancer markers present in the relevant samples. This information may be helpful in determining applicable targets for diagnostic and therapeutic purposes.

For clarity, TABLE 5 presents a tabular listing of the results in FIG. 8. TABLE 5 and FIG. 8 show that PCR Reduction assays using the 59 cancer detection reagents of TABLE 1 are sensitive enough to detect their representative cancer markers in metastasized cancer cells from blood samples. This sensitivity may results from the one-to-many genetic association of the cancer markers, and thus in many instances, once a blood sample is provided, there will be no further need for tissue samples or biopsies to facilitate cancer pathology analysis.

Cancer detection reagents of the present invention are generally designed to detect mutations that are exclusive to cancer cells, not specific tumors. It has been shown that the cancer detection reagents can detect cancer markers in cells circulating in the blood. So, one would expect PCR Reduction tests for a tumor tissue sample and a blood sample from the same subject to show an increased number of cancer markers in the blood. In fact, any cancer marker profile from a tissue sample alone will likely be inferior to a blood sample because the tissue sample profile is actually a profile for the single, biopsied tumor, and not the subject's cancer in general. This can be seen somewhat in TABLE 5 which shows an increased number of mutations from the blood sample of patient H versus the tissue sample of patient R.

However, to more clearly show the superiority of blood samples over tissue samples, a side by side PCR Reduction assay was conducted using both types of samples from the same cancer subject. The results of this assay for samples from patient R are shown in TABLE 5. Substantially more cancer detection reagents yielded positive results for the blood sample than for the tissue sample alone for patient R.

Further, the tissue sample was obtained in March, 2004 and the blood sample was obtained in December, 2004. In the interim the subject underwent extensive cancer therapies. The high number of mutations in the December blood sample not only reflects the ineffectiveness of the cancer therapies (as confirmed by standard clinical observations), it also reflects the high level of cancer cell traffic in the blood of patient R. This heavy traffic can most likely be correlated with highly active cancer, as is also indicated by patient R's failure to respond to traditional treatment and her continually deteriorating condition.

### Example 9

### Microarrays

Blood samples may also be analyzed using microarrays containing single stranded DNA molecules having the sequences of cancer markers. These DNA molecules represent yet another type of cancer detection reagent. Such microarrays may be created using known techniques, but incorporating the new cancer markers. For example, a microarray for detecting cancer markers 3 and 5-87 may contain single stranded DNA from either strand of the oligos listed in TABLE 1. Blood samples may then be applied to the microarray and the microarray read using known methods to reveal which cancer markers are exhibited by a particular subject's tumors. To affirm the viability of this approach, blood samples may be compared with tumor samples to see if an increased number of cancer markers are observed in the blood samples, as expected. Additionally, results may be compared with those obtained using PCR. It is expected that the results using a microarray should be identical or nearly identical, with any differences explainable by differing sensitivities of the methods.

In particular, microarrays may be created using the standard procedures of microarray manufacturers such as Affymetrix (California).

Although the present invention and its advantages have been described in detail, it should be understood that various changes, substitutions and alternations can be made herein without departing from the spirit and scope of the invention as defined by the following claims.

1. A cancer detection reagent set comprising cancer detection reagents corresponding to at least 10 general cancer markers.

2. The cancer detection reagent set of claim 1, comprising cancer detection reagents corresponding to at least 50 general cancer markers.

3. The cancer detection reagent set of claim 1, comprising cancer detection reagents corresponding to at least 100 general cancer markers.

4. A cancer detection reagent set comprising cancer detection reagents corresponding to at least 10 colon cancer markers.

5. The cancer detection reagent set of claim 4, comprising cancer detection reagents corresponding to at least 50 colon cancer markers.

6. The cancer detection reagent set of claim 4, comprising cancer detection reagents corresponding to at least 100 colon cancer markers.

7. A cancer detection reagent set comprising cancer detection reagents corresponding to at least 10 lung cancer markers.

8. The cancer detection reagent set of claim 7, comprising cancer detection reagents corresponding to at least 50 lung cancer markers.

9. The cancer detection reagent set of claim 7, comprising cancer detection reagents corresponding to at least 100 lung cancer markers.

10. A cancer detection reagent set comprising cancer detection reagents corresponding to at least 10 lymph cancer markers.

11. The cancer detection reagent set of claim 10, comprising cancer detection reagents corresponding to at least 50 lymph cancer markers.

12. The cancer detection reagent set of claim 10, comprising cancer detection reagents corresponding to at least 100 lymph cancer markers.

13. A cancer detection reagent set comprising cancer detection reagents corresponding to at least 10 breast cancer markers.

14. The cancer detection reagent set of claim 13, comprising cancer detection reagents corresponding to at least 50 breast cancer markers.

15. The cancer detection reagent set of claim 13, comprising cancer detection reagents corresponding to at least 100 breast cancer markers.

16. The cancer detection reagent set of claim 4, comprising at least 50 cancer markers selected from the group consisting of cancer markers 3 and 55-66, and any combinations thereof.

17. The cancer detection reagent set of claim 1, further comprising a microarray having the reagent set.

18. The cancer detection reagent set of claim 17, wherein the cancer markers are selected from the group consisting of: colon cancer markers, lung cancer markers, lymph cancer markers, breast cancer markers, and any combinations thereof.

19. The cancer detection reagent set of claim 1, further comprising a PCR kit having the cancer markers.

20. The cancer detection reagent set of claim 19 wherein the cancer markers are selected from the group consisting of: colon cancer markers, lung cancer markers, lymph cancer markers, breast cancer markers, and any combinations thereof.

21. A method of detecting a cancer marker in a sample comprising:
extracting mRNA from the sample;
creating cDNA from the mRNA;
performing at least 10 separate PCR reduction reactions using the cDNA as a template and at least 10 different single primers, with a different single primer in each separate PCR reduction reaction; and
analyzing the product of each of the 10 separate PCR reduction reactions to determine the presence or absence of primer-amplified DNA molecules, where presence of primer-amplified DNA molecules in any PCR reduction reaction indicates the presence of a cancer marker;
wherein the 10 different single primers each have an antisense sequence corresponding to a different cancer marker.

22. The method of claim 21, where the sample comprises peripheral blood.

23. A method of detecting cancer markers in a sample comprising:
isolating sample nucleic acids from the sample;
placing the sample nucleic acids on a microarray having DNA molecules operable to detect and distinguish at least 10 corresponding cancer markers under conditions sufficient to allow detectable and specific binding of sample nucleic acids to complementary DNA molecules of the microarray; and
detecting binding of sample nucleic acids to the microarray,
wherein binding of sample nucleic acids to a DNA molecule on the microarray indicates the presence of a cancer marker corresponding to that DNA molecule in the sample.

24. The method of claim 23, wherein the sample comprises peripheral blood.


SEQUENCE LISTING (398 sequences)

SEQ ID NO: 1 - DNA, 17 bp, Homo sapiens
aagggggttccttgggc

SEQ ID NO: 2 - DNA, 17 bp, Homo sapiens
ggcctgccagaagcaca

SEQ ID NO: 3 - DNA, 17 bp, Homo sapiens
gccgattaacaccagcc

SEQ ID NO: 4 - DNA, 17 bp, Homo sapiens
cgattaaccaccggcct

SEQ ID NO: 5 - DNA, 17 bp, Homo sapiens
ttgaaccctaggcatgt

SEQ ID NO: 6 - DNA, 17 bp, Homo sapiens
cctgagcaaacctgagc

SEQ ID NO: 7 - DNA, 17 bp, Homo sapiens
cgcatgcgtggccacca

SEQ ID NO: 8 - DNA, 17 bp, Homo sapiens
ccaactggatcccaggt

SEQ ID NO: 9 - DNA, 17 bp, Homo sapiens
cggatgtccctgctggg

SEQ ID NO: 10 - DNA, 17 bp, Homo sapiens
gccgattcacacccagc

SEQ ID NO: 11 - DNA, 16 bp, Homo sapiens
gcctcgtacctagccg

SEQ ID NO: 12 - DNA, 17 bp, Homo sapiens
cgcctcggccgattaac

SEQ ID NO: 13 - DNA, 17 bp, Homo sapiens
ggccgatttacacccgg

SEQ ID NO: 14 - DNA, 17 bp, Homo sapiens
tcggccgattaacccca

SEQ ID NO: 15 - DNA, 17 bp, Homo sapiens
ccgattaacaccggcct

SEQ ID NO: 16 - DNA, 18 bp, Homo sapiens
gctgttgtcatacttgct

SEQ ID NO: 17 - DNA, 17 bp, Homo sapiens
ccacgtgatgtagactg

SEQ ID NO: 18 - DNA, 17 bp, Homo sapiens
cccagcctcgtacctag

SEQ ID NO: 19 - DNA, 18 bp, Homo sapiens
cagcctctacctagcctt

SEQ ID NO: 20 - DNA, 16 bp, Homo sapiens
caccggcctcgtacct

SEQ ID NO: 21 - DNA, 17 bp, Homo sapiens
gcccaaggaaccccctt

SEQ ID NO: 22 - DNA, 17 bp, Homo sapiens
gctcaggtttgctcagg

SEQ ID NO: 23 - DNA, 17 bp, Homo sapiens
tgtgcttctggcaggcc

SEQ ID NO: 24 - DNA, 25 bp, Homo sapiens
acctgggatccagttggaggacggc

SEQ ID NO: 25 - DNA, 25 bp, Homo sapiens
gccgtcctccaactggatcccaggt

SEQ ID NO: 26 - DNA, 17 bp, Homo sapiens
tggtggccacgcatgcg

SEQ ID NO: 27 - DNA, 17 bp, Homo sapiens
cccagcagggacatccg

SEQ ID NO: 28 - DNA, 18 bp, Homo sapiens
ggctaggtacgaggctgg

SEQ ID NO: 29 - DNA, 18 bp, Homo sapiens
ccagcctcgtacctagcc

SEQ ID NO: 30 - DNA, 22 bp, Homo sapiens
ggctggtgttaatcggccgagg

SEQ ID NO: 31 - DNA, 22 bp, Homo sapiens
cctcggccgattaacaccagcc

SEQ ID NO: 32 - DNA, 19 bp, Homo sapiens
gggggtgaatcggccgagg

SEQ ID NO: 33 - DNA, 19 bp, Homo sapiens
cctcggccgattcaccccc

SEQ ID NO: 34 - DNA, 22 bp, Homo sapiens
gctgggtgtgaatcggccgagg

SEQ ID NO: 35 - DNA, 22 bp, Homo sapiens
cctcggccgattcacacccagc

SEQ ID NO: 36 - DNA, 19 bp, Homo sapiens
aggtacgaggccgggtgtt

SEQ ID NO: 37 - DNA, 19 bp, Homo sapiens
aacacccggcctcgtacct

SEQ ID NO: 38 - DNA, 17 bp, Homo sapiens
gtgttaatcggccgagg

SEQ ID NO: 39 - DNA, 17 bp, Homo sapiens
cctcggccgattaacac

SEQ ID NO: 40 - DNA, 17 bp, Homo sapiens
agatgggtaccaactgt

SEQ ID NO: 41 - DNA, 17 bp, Homo sapiens
acagttggtacccatct

SEQ ID NO: 42 - DNA, 22 bp, Homo sapiens
cggctaggtacgaggctggggt

SEQ ID NO: 43 - DNA, 22 bp, Homo sapiens
accccagcctcgtacctagccg

SEQ ID NO: 44 - DNA, 24 bp, Homo sapiens
gaggcgggtgtgaatcggccgagg

SEQ ID NO: 45 - DNA, 24 bp, Homo sapiens
cctcggccgattcacacccgcctc

SEQ ID NO: 46 - DNA, 17 bp, Homo sapiens
aggtacgaggccggtgt

SEQ ID NO: 47 - DNA, 17 bp, Homo sapiens
acaccggcctcgtacct

SEQ ID NO: 48 - DNA, 18 bp, Homo sapiens
gttaatcggccgaggcgc

SEQ ID NO: 49 - DNA, 18 bp, Homo sapiens
gcgcctcggccgattaac

SEQ ID NO: 50 - DNA, 17 bp, Homo sapiens
agaccaacagagttcgg

SEQ ID NO: 51 - DNA, 17 bp, Homo sapiens
ccgaactctgttggtct

SEQ ID NO: 52 - DNA, 19 bp, Homo sapiens
tggcttcgtgtcccatgca

SEQ ID NO: 53 - DNA, 19 bp, Homo sapiens
tgcatgggacacgaagcca

SEQ ID NO: 54 - DNA, 19 bp, Homo sapiens
ccgggtgtaaatcggccga

SEQ ID NO: 55 - DNA, 19 bp, Homo sapiens
tcggccgatttacacccgg

SEQ ID NO: 56 - DNA, 19 bp, Homo sapiens
gccggtgtgaatcggccga

SEQ ID NO: 57 - DNA, 19 bp, Homo sapiens
tcggccgattcacaccggc

SEQ ID NO: 58 - DNA, 20 bp, Homo sapiens
tcatgatggtgtatcgatga

SEQ ID NO: 59 - DNA, 20 bp, Homo sapiens
tcatcgatacaccatcatga

SEQ ID NO: 60 - DNA, 20 bp, Homo sapiens
gctcggtgttaatcggccga

SEQ ID NO: 61 - DNA, 20 bp, Homo sapiens
tcggccgattaacaccgagc

SEQ ID NO: 62 - DNA, 19 bp, Homo sapiens
tggggttaatcggccgagg

SEQ ID NO: 63 - DNA, 19 bp, Homo sapiens
cctcggccgattaacccca

SEQ ID NO: 64 - DNA, 21 bp, Homo sapiens
aggccggtgttaatcggccga

SEQ ID NO: 65 - DNA, 21 bp, Homo sapiens
tcggccgattaacaccggcct

SEQ ID NO: 66 - DNA, 19 bp, Homo sapiens
tggtgaatcggccgagggt

SEQ ID NO: 67 - DNA, 19 bp, Homo sapiens
accctcggccgattcacca

SEQ ID NO: 68 - DNA, 18 bp, Homo sapiens
agcaagtatgacaacagc

SEQ ID NO: 69 - DNA, 18 bp, Homo sapiens
gctgttgtcatacttgct

SEQ ID NO: 70 - DNA, 17 bp, Homo sapiens
cttaaaccaagctagcc

SEQ ID NO: 71 - DNA, 17 bp, Homo sapiens
ggctagcttggtttaag

SEQ ID NO: 72 - DNA, 17 bp, Homo sapiens
cagtctacatcacgtgg

SEQ ID NO: 73 - DNA, 17 bp, Homo sapiens
ccacgtgatgtagactg

SEQ ID NO: 74 - DNA, 18 bp, Homo sapiens
aatctcctgttacactca

SEQ ID NO: 75 - DNA, 18 bp, Homo sapiens
tgagtgtaacaggagatt

SEQ ID NO: 76 - DNA, 17 bp, Homo sapiens
gcccaaggaaccccctt

SEQ ID NO: 77 - DNA, 17 bp, Homo sapiens
aagggggttccttgggc

SEQ ID NO: 78 - DNA, 18 bp, Homo sapiens
ggctaggacgaggccggg

SEQ ID NO: 79 - DNA, 18 bp, Homo sapiens
cccggcctcgtcctagcc

SEQ ID NO: 80 - DNA, 17 bp, Homo sapiens
gagaaggttcccgggaa

SEQ ID NO: 81 - DNA, 17 bp, Homo sapiens
ttcccgggaaccttctc

SEQ ID NO: 82 - DNA, 17 bp, Homo sapiens
gtgttactcggccgagg

SEQ ID NO: 83 - DNA, 17 bp, Homo sapiens
cctcggccgagtaacac

SEQ ID NO: 84 - DNA, 18 bp, Homo sapiens
ttgaatcggccgagggtg

SEQ ID NO: 85 - DNA, 18 bp, Homo sapiens
caccctcggccgattcaa

SEQ ID NO: 86 - DNA, 17 bp, Homo sapiens
gccgggtggtgaatcgg

SEQ ID NO: 87 - DNA, 17 bp, Homo sapiens
ccgattcaccacccggc

SEQ ID NO: 88 - DNA, 17 bp, Homo sapiens
gccggtggttaatcggc

SEQ ID NO: 89 - DNA, 17 bp, Homo sapiens
gccgattaaccaccggc

SEQ ID NO: 90 - DNA, 17 bp, Homo sapiens
gggcgcagcgacatcag

SEQ ID NO: 91 - DNA, 17 bp, Homo sapiens
ctgatgtcgctgcgccc

SEQ ID NO: 92 - DNA, 18 bp, Homo sapiens
gctattagcagattgtgt

SEQ ID NO: 93 - DNA, 18 bp, Homo sapiens
acacaatctgctaatagc

SEQ ID NO: 94 - DNA, 22 bp, Homo sapiens
tgttaatctcctgttacactca

SEQ ID NO: 95 - DNA, 22 bp, Homo sapiens
tgagtgtaacaggagattaaca

SEQ ID NO: 96 - DNA, 17 bp, Homo sapiens
ccaccgcaccgttggcc

SEQ ID NO: 97 - DNA, 17 bp, Homo sapiens
ggccaacggtgcggtgg

SEQ ID NO: 98 - DNA, 17 bp, Homo sapiens
acctggagccctctgat

SEQ ID NO: 99 - DNA, 17 bp, Homo sapiens
atcagagggctccaggt

SEQ ID NO: 100 - DNA, 18 bp, Homo sapiens
tcagacaaacacagatcg

SEQ ID NO: 101 - DNA, 18 bp, Homo sapiens
cgatctgtgtttgtctga

SEQ ID NO: 102 - DNA, 21 bp, Homo sapiens
gagaatactgattgagaccta

SEQ ID NO: 103 - DNA, 21 bp, Homo sapiens
taggtctcaatcagtattctc

SEQ ID NO: 104 - DNA, 17 bp, Homo sapiens
ccagccagcacccaggc

SEQ ID NO: 105 - DNA, 17 bp, Homo sapiens
gcctgggtgctggctgg

SEQ ID NO: 106 - DNA, 17 bp, Homo sapiens
tagaccaacagagttcc

SEQ ID NO: 107 - DNA, 17 bp, Homo sapiens
ggaactctgttggtcta

SEQ ID NO: 108 - DNA, 21 bp, Homo sapiens
ctaggtacgaggctgggtttt

SEQ ID NO: 109 - DNA, 21 bp, Homo sapiens
aaaacccagcctcgtacctag

SEQ ID NO: 110 - DNA, 21 bp, Homo sapiens
cgaggcgggtgttaatcggcc

SEQ ID NO: 111 - DNA, 21 bp, Homo sapiens
ggccgattaacacccgcctcg

SEQ ID NO: 112 - DNA, 18 bp, Homo sapiens
aaggctaggtagaggctg

SEQ ID NO: 113 - DNA, 18 bp, Homo sapiens
cagcctctacctagcctt

SEQ ID NO: 114 - DNA, 17 bp, Homo sapiens
catggccatgctgtgca

SEQ ID NO: 115 - DNA, 17 bp, Homo sapiens
tgcacagcatggccatg

SEQ ID NO: 116 - DNA, 28 bp, Homo sapiens
aggtacgaggccggtgttaatcggccga

SEQ ID NO: 117 - DNA, 28 bp, Homo sapiens
tcggccgattaacaccggcctcgtacct

SEQ ID NO: 118 - DNA, 17 bp, Homo sapiens
tgctgccctcaatggtc

SEQ ID NO: 119 - DNA, 17 bp, Homo sapiens
gaccattgagggcagca

SEQ ID NO: 120 - DNA, 24 bp, Homo sapiens
aggccggtggttaatcggccgagg

SEQ ID NO: 121 - DNA, 24 bp, Homo sapiens
cctcggccgattaaccaccggcct

SEQ ID NO: 122 - DNA, 24 bp, Homo sapiens
gaggccggtggttaatcggccgag

SEQ ID NO: 123 - DNA, 24 bp, Homo sapiens
ctcggccgattaaccaccggcctc

SEQ ID NO: 124 - DNA, 22 bp, Homo sapiens
gctaggtacgaggctgggtttt

SEQ ID NO: 125 - DNA, 22 bp, Homo sapiens
aaaacccagcctcgtacctagc

SEQ ID NO: 126 - DNA, 19 bp, Homo sapiens
aacatacggctaggtacga

SEQ ID NO: 127 - DNA, 19 bp, Homo sapiens
tcgtacctagccgtatgtt

SEQ ID NO: 128 - DNA, 18 bp, Homo sapiens
ggtggtaatcggacgagg

SEQ ID NO: 129 - DNA, 18 bp, Homo sapiens
cctcgtccgattaccacc

SEQ ID NO: 130 - DNA, 17 bp, Homo sapiens
gggtgatcggacgaggc

SEQ ID NO: 131 - DNA, 17 bp, Homo sapiens
gcctcgtccgatcaccc

SEQ ID NO: 132 - DNA, 17 bp, Homo sapiens
acatgcctagggttcaa

SEQ ID NO: 133 - DNA, 62 bp, Homo sapiens
tgccctccacaggactctccctactgcctgagcaaacctgagcctcccggcagacccaccca

SEQ ID NO: 134 - DNA, 61 bp, Homo sapiens
tgccctccacagactctccctactgcctgagcaaacctgagcgtcccggcagacccaccca

SEQ ID NO: 135 - DNA, 62 bp, Homo sapiens; identical to SEQ ID NO: 133

SEQ ID NO: 136 - DNA, 62 bp, Homo sapiens
tgccctccacaggactctccctactgcctgagcaaacctgagcgtcccggcagacccaccca

SEQ ID NO: 137 - DNA, 62 bp, Homo sapiens; identical to SEQ ID NO: 136

SEQ ID NO: 138 - DNA, 63 bp, Homo sapiens
tgccctccacaggactctccctactgcctgagcaaacctgagcgctcccggcagacccaccca

SEQ ID NO: 139 - DNA, 63 bp, Homo sapiens
tgccctccacagtactctccctactgcctgagcaaacctgagcgctcccggcagacccaccca

SEQ ID NO: 140 - DNA, 55 bp, Homo sapiens
tgcctcacagatctcctactcctgagcaaacctgagcgtccggcagacccaccca

SEQ ID NO: 141 - DNA, 63 bp, Homo sapiens
tgccctccacaggactctccctactgcctgagcaaacctgaggcctcccggcagacccaccca

SEQ ID NO: 142 - DNA, 52 bp, Homo sapiens
ctggcctgagaagttctgcacatgcgtggcaccatttcctgtgaacacttgc

SEQ ID NO: 143 - DNA, 52 bp, Homo sapiens
ccagcccgggaaggtttgcgcgcgtgtggcaccatttcccatgaacacccat

SEQ ID NO: 144 - DNA, 52 bp, Homo sapiens
ctggctcgggaagttctgcgcatgcgtggcaccatttcccgtgaacacccat

SEQ ID NO: 145 - DNA, 52 bp, Homo sapiens
ctggctcgggaagttctgcgcatgcgtggcaccatttctcgtgaacacctat

SEQ ID NO: 146 - DNA, 52 bp, Homo sapiens
ctggctcgggaagttctgcgcatgcgtggcaccgtttcccgtgaacacccgt

SEQ ID NO: 147 - DNA, 52 bp, Homo sapiens
ctggcttgggaagttctgcgcatgcatggcaccatttcccgtgaacacccat

SEQ ID NO: 148 - DNA, 32 bp, Homo sapiens
aagttctgcgcatgcgtggcaccatttctcgt

SEQ ID NO: 149 - DNA, 32 bp, Homo sapiens
aagttctgcgcatgcgtggcaccatttcccgt

SEQ ID NO: 150 - DNA, 32 bp, Homo sapiens; identical to SEQ ID NO: 149

SEQ ID NO: 151 - DNA, 32 bp, Homo sapiens
aagttctgcgcatgcgtggcaccatttcctgt

SEQ ID NO: 152 - DNA, 30 bp, Homo sapiens
gtactgtgcatgcgtggcaccatttcccgt

SEQ ID NO: 153 - DNA, 30 bp, Homo sapiens
gttctgtgcatgcgtggcaccatttcccgt

SEQ ID NO: 154 - DNA, 32 bp, Homo sapiens
aagttctgcacatgcgtggcaccatttcctgt

SEQ ID NO: 155 - DNA, 32 bp, Homo sapiens
aagttctgcgcatgtgtggcaccatttcccgt

SEQ ID NO: 156 - DNA, 30 bp, Homo sapiens
gttctgtgcatgcttggcaccatttcctgt

SEQ ID NO: 157 - DNA, 32 bp, Homo sapiens
aagttctgcacatgtgtggcaccatttcctgt

SEQ ID NO: 158 - DNA, 32 bp, Homo sapiens
aagttttgcgcatgcgtgacaccatttcccat

SEQ ID NO: 159 - DNA, 22 bp, Homo sapiens
cctctgttaatctcctgttaca

SEQ ID NO: 160 - DNA, 19 bp, Homo sapiens
cctctgttaatctcctgtt

SEQ ID NO: 161 - DNA, 17 bp, Homo sapiens
gcccaaggaaccccctt

SEQ ID NO: 162 - DNA, 36 bp, Homo sapiens
gactgaatgcacccaatatccgacctggctgcgtgt

SEQ ID NO: 163 - DNA, 19 bp, Homo sapiens
gactgaatgcacccaatat

SEQ ID NO: 164 - DNA, 25 bp, Homo sapiens
caccctctgttaatctcctgttaca

SEQ ID NO: 165 - DNA, 18 bp, Homo sapiens
caccctctgttaatctcc

SEQ ID NO: 166 - DNA, 17 bp, Homo sapiens
gctcaggtttgctcagg

SEQ ID NO: 167 - DNA, 17 bp, Homo sapiens
tgtgcttctggcaggcc

SEQ ID NO: 168 - DNA, 25 bp, Homo sapiens
acctgggatccagttggaggacggc

SEQ ID NO: 169 - DNA, 17 bp, Homo sapiens
acctgggatccagttgg

SEQ ID NO: 170 - DNA, 17 bp, Homo sapiens
cgcatgcgtggccacca

SEQ ID NO: 171 - DNA, 17 bp, Homo sapiens
cccagcagggacatccg

SEQ ID NO: 172 - DNA, 18 bp, Homo sapiens
ggctaggtacgaggctgg

SEQ ID NO: 173 - DNA, 17 bp, Homo sapiens
ggctaggtacgaggctg

SEQ ID NO: 174 - DNA, 22 bp, Homo sapiens
ggctggtgttaatcggccgagg

SEQ ID NO: 175 - DNA, 17 bp, Homo sapiens
ggctggtgttaatcggc

SEQ ID NO: 176 - DNA, 19 bp, Homo sapiens
gggggtgaatcggccgagg

SEQ ID NO: 177 - DNA, 16 bp, Homo sapiens
gggggtgaatcggccg

SEQ ID NO: 178 - DNA, 22 bp, Homo sapiens
gctgggtgtgaatcggccgagg

SEQ ID NO: 179 - DNA, 17 bp, Homo sapiens
gctgggtgtgaatcggc

SEQ ID NO: 180 - DNA, 19 bp, Homo sapiens
aggtacgaggccgggtgtt

SEQ ID NO: 181 - DNA, 16 bp, Homo sapiens
aggtacgaggccgggt

SEQ ID NO: 182 - DNA, 17 bp, Homo sapiens
gtgttaatcggccgagg

SEQ ID NO: 183 - DNA, 17 bp, Homo sapiens
agatgggtaccaactgt

SEQ ID NO: 184 - DNA, 22 bp, Homo sapiens
cggctaggtacgaggctggggt

SEQ ID NO: 185 - DNA, 16 bp, Homo sapiens
cggctaggtacgaggc

SEQ ID NO: 186 - DNA, 24 bp, Homo sapiens
gaggcgggtgtgaatcggccgagg

SEQ ID NO: 187 - DNA, 17 bp, Homo sapiens
gaggcgggtgtgaatcg

SEQ ID NO: 188 - DNA, 17 bp, Homo sapiens
aggtacgaggccggtgt

SEQ ID NO: 189 - DNA, 18 bp, Homo sapiens
gttaatcggccgaggcgc

SEQ ID NO: 190 - DNA, 17 bp, Homo sapiens
gttaatcggccgaggcg

SEQ ID NO: 191 - DNA, 17 bp, Homo sapiens
agaccaacagagttcgg

SEQ ID NO: 192 - DNA, 19 bp, Homo sapiens
tggcttcgtgtcccatgca

SEQ ID NO: 193 - DNA, 17 bp, Homo sapiens
tggcttcgtgtcccatg

SEQ ID NO: 194 - DNA, 19 bp, Homo sapiens
ccgggtgtaaatcggccga

SEQ ID NO: 195 - DNA, 17 bp, Homo sapiens
ccgggtgtaaatcggcc

SEQ ID NO: 196 - DNA, 19 bp, Homo sapiens
gccggtgtgaatcggccga

SEQ ID NO: 197 - DNA, 16 bp, Homo sapiens
gccggtgtgaatcggc

SEQ ID NO: 198 - DNA, 20 bp, Homo sapiens
tcatgatggtgtatcgatga

SEQ ID NO: 199 - DNA, 20 bp, Homo sapiens
gctcggtgttaatcggccga

SEQ ID NO: 200 - DNA, 17 bp, Homo sapiens
gctcggtgttaatcggc

SEQ ID NO: 201 - DNA, 19 bp, Homo sapiens
tggggttaatcggccgagg

SEQ ID NO: 202 - DNA, 17 bp, Homo sapiens
tggggttaatcggccga

SEQ ID NO: 203 - DNA, 21 bp, Homo sapiens
aggccggtgttaatcggccga

SEQ ID NO: 204 - DNA, 17 bp, Homo sapiens
aggccggtgttaatcgg

SEQ ID NO: 205 - DNA, 19 bp, Homo sapiens
tggtgaatcggccgagggt

SEQ ID NO: 206 - DNA, 17 bp, Homo sapiens
tggtgaatcggccgagg

SEQ ID NO: 207 - DNA, 18 bp, Homo sapiens
agcaagtatgacaacagc

SEQ ID NO: 208 - DNA, 17 bp, Homo sapiens
cttaaaccaagctagcc

SEQ ID NO: 209 - DNA, 17 bp, Homo sapiens
cagtctacatcacgtgg

SEQ ID NO: 210 - DNA, 18 bp, Homo sapiens
aatctcctgttacactca

SEQ ID NO: 211 - DNA, 17 bp, Homo sapiens
gcccaaggaaccccctt

SEQ ID NO: 212 - DNA, 18 bp, Homo sapiens
ggctaggacgaggccggg

SEQ ID NO: 213 - DNA, 16 bp, Homo sapiens
ggctaggacgaggccg

SEQ ID NO: 214 - DNA, 17 bp, Homo sapiens
gagaaggttcccgggaa

SEQ ID NO: 215 - DNA, 17 bp, Homo sapiens
gtgttactcggccgagg

SEQ ID NO: 216 - DNA, 18 bp, Homo sapiens
ttgaatcggccgagggtg

SEQ ID NO: 217 - DNA, 17 bp, Homo sapiens
gccgggtggtgaatcgg

SEQ ID NO: 218 - DNA, 17 bp, Homo sapiens
gccggtggttaatcggc

SEQ ID NO: 219 - DNA, 17 bp, Homo sapiens
gggcgcagcgacatcag

SEQ ID NO: 220 - DNA, 18 bp, Homo sapiens
gctattagcagattgtgt

SEQ ID NO: 221 - DNA, 22 bp, Homo sapiens
tgttaatctcctgttacactca

SEQ ID NO: 222 - DNA, 20 bp, Homo sapiens
tgttaatctcctgttacact

SEQ ID NO: 223 - DNA, 17 bp, Homo sapiens
ccaccgcaccgttggcc

SEQ ID NO: 224 - DNA, 16 bp, Homo sapiens
ccaccgcaccgttggc

SEQ ID NO: 225 - DNA, 17 bp, Homo sapiens
acctggagccctctgat

SEQ ID NO: 226 - DNA, 18 bp, Homo sapiens
tcagacaaacacagatcg

SEQ ID NO: 227 - DNA, 21 bp, Homo sapiens
gagaatactgattgagaccta

SEQ ID NO: 228 - DNA, 17 bp, Homo sapiens
ccagccagcacccaggc

SEQ ID NO: 229 - DNA, 16 bp, Homo sapiens
ccagccagcacccagg

SEQ ID NO: 230 - DNA, 17 bp, Homo sapiens
tagaccaacagagttcc

SEQ ID NO: 231 - DNA, 21 bp, Homo sapiens
ctaggtacgaggctgggtttt

SEQ ID NO: 232 - DNA, 17 bp, Homo sapiens
ctaggtacgaggctggg

SEQ ID NO: 233 - DNA, 21 bp, Homo sapiens
cgaggcgggtgttaatcggcc

SEQ ID NO: 234 - DNA, 17 bp, Homo sapiens
cgaggcgggtgttaatc

SEQ ID NO: 235 - DNA, 18 bp, Homo sapiens
aaggctaggtagaggctg

SEQ ID NO: 236 - DNA, 17 bp, Homo sapiens
catggccatgctgtgca

SEQ ID NO: 237 - DNA, 28 bp, Homo sapiens
aggtacgaggccggtgttaatcggccga

SEQ ID NO: 238 - DNA, 16 bp, Homo sapiens
aggtacgaggccggtg

SEQ ID NO: 239 - DNA, 21 bp, Homo sapiens
tgcaccacaagcaaacaggcc

SEQ ID NO: 240 - DNA, 18 bp, Homo sapiens
tgcaccacaagcaaacag

SEQ ID NO: 241 - DNA, 17 bp, Homo sapiens
tgctgccctcaatggtc

SEQ ID NO: 242 - DNA, 24 bp, Homo sapiens
aggccggtggttaatcggccgagg

SEQ ID NO: 243 - DNA, 17 bp, Homo sapiens
aggccggtggttaatcg

SEQ ID NO: 244 - DNA, 24 bp, Homo sapiens
gaggccggtggttaatcggccgag

SEQ ID NO: 245 - DNA, 17 bp, Homo sapiens
gaggccggtggttaatc

SEQ ID NO: 246 - DNA, 22 bp, Homo sapiens
gctaggtacgaggctgggtttt

SEQ ID NO: 247 - DNA, 17 bp, Homo sapiens
gctaggtacgaggctgg

SEQ ID NO: 248 - DNA, 19 bp, Homo sapiens
aacatacggctaggtacga

SEQ ID NO: 249 - DNA, 18 bp, Homo sapiens
ggtggtaatcggacgagg

SEQ ID NO: 250 - DNA, 17 bp, Homo sapiens
gggtgatcggacgaggc

SEQ ID NO: 251 - DNA, 17 bp, Homo sapiens
acatgcctagggttcaa

SEQ ID NO: 252 - DNA, 22 bp, Homo sapiens
tgtaacaggagattaacagagg

SEQ ID NO: 253 - DNA, 19 bp, Homo sapiens
aacaggagattaacagagg

SEQ ID NO: 254 - DNA, 17 bp, Homo sapiens
aagggggttccttgggc

SEQ ID NO: 255 - DNA, 36 bp, Homo sapiens
acacgcagccaggtcggatattgggtgcattcagtc

SEQ ID NO: 256 - DNA, 19 bp, Homo sapiens
atattgggtgcattcagtc

SEQ ID NO: 257 - DNA, 25 bp, Homo sapiens
tgtaacaggagattaacagagggtg

SEQ ID NO: 258 - DNA, 18 bp, Homo sapiens
ggagattaacagagggtg

SEQ ID NO: 259 - DNA, 17 bp, Homo sapiens
cctgagcaaacctgagc

SEQ ID NO: 260 - DNA, 17 bp, Homo sapiens
ggcctgccagaagcaca

SEQ ID NO: 261 - DNA, 25 bp, Homo sapiens
gccgtcctccaactggatcccaggt

SEQ ID NO: 262 - DNA, 17 bp, Homo sapiens
ccaactggatcccaggt

SEQ ID NO: 263 - DNA, 17 bp, Homo sapiens
tggtggccacgcatgcg

SEQ ID NO: 264 - DNA, 17 bp, Homo sapiens
cggatgtccctgctggg

SEQ ID NO: 265 - DNA, 18 bp, Homo sapiens
ccagcctcgtacctagcc

SEQ ID NO: 266 - DNA, 17 bp, Homo sapiens
cagcctcgtacctagcc

SEQ ID NO: 267 - DNA, 22 bp, Homo sapiens
cctcggccgattaacaccagcc

SEQ ID NO: 268 - DNA, 17 bp, Homo sapiens
gccgattaacaccagcc

SEQ ID NO: 269 - DNA, 19 bp, Homo sapiens
cctcggccgattcaccccc

SEQ ID NO: 270 - DNA, 16 bp, Homo sapiens
cggccgattcaccccc

SEQ ID NO: 271 - DNA, 22 bp, Homo sapiens
cctcggccgattcacacccagc

SEQ ID NO: 272 - DNA, 17 bp, Homo sapiens
gccgattcacacccagc

SEQ ID NO: 273 - DNA, 19 bp, Homo sapiens
aacacccggcctcgtacct

SEQ ID NO: 274 - DNA, 16 bp, Homo sapiens
acccggcctcgtacct

SEQ ID NO: 275 - DNA, 17 bp, Homo sapiens
cctcggccgattaacac

SEQ ID NO: 276 - DNA, 17 bp, Homo sapiens
acagttggtacccatct

SEQ ID NO: 277 - DNA, 22 bp, Homo sapiens
accccagcctcgtacctagccg

SEQ ID NO: 278 - DNA, 16 bp, Homo sapiens
gcctcgtacctagccg

SEQ ID NO: 279 - DNA, 24 bp, Homo sapiens
cctcggccgattcacacccgcctc

SEQ ID NO: 280 - DNA, 17 bp, Homo sapiens
cgattcacacccgcctc

SEQ ID NO: 281 - DNA, 17 bp, Homo sapiens
acaccggcctcgtacct

SEQ ID NO: 282 - DNA, 18 bp, Homo sapiens
gcgcctcggccgattaac

SEQ ID NO: 283 - DNA, 17 bp, Homo sapiens
cgcctcggccgattaac

SEQ ID NO: 284 - DNA, 17 bp, Homo sapiens
ccgaactctgttggtct

SEQ ID NO: 285 - DNA, 19 bp, Homo sapiens
tgcatgggacacgaagcca

SEQ ID NO: 286 - DNA, 17 bp, Homo sapiens
catgggacacgaagcca

SEQ ID NO: 287 - DNA, 19 bp, Homo sapiens
tcggccgatttacacccgg

SEQ ID NO: 288 - DNA, 17 bp, Homo sapiens
ggccgatttacacccgg

SEQ ID NO: 289 - DNA, 19 bp, Homo sapiens
tcggccgattcacaccggc

SEQ ID NO: 290 - DNA, 16 bp, Homo sapiens
gccgattcacaccggc

SEQ ID NO: 291 - DNA, 20 bp, Homo sapiens
tcatcgatacaccatcatga

SEQ ID NO: 292 - DNA, 20 bp, Homo sapiens
tcggccgattaacaccgagc

SEQ ID NO: 293 - DNA, 17 bp, Homo sapiens
gccgattaacaccgagc

SEQ ID NO: 294 - DNA, 19 bp, Homo sapiens
cctcggccgattaacccca

SEQ ID NO: 295 - DNA, 17 bp, Homo sapiens
tcggccgattaacccca

SEQ ID NO: 296 - DNA, 21 bp, Homo sapiens
tcggccgattaacaccggcct

SEQ ID NO: 297 - DNA, 17 bp, Homo sapiens
ccgattaacaccggcct

SEQ ID NO: 298 - DNA, 19 bp, Homo sapiens
accctcggccgattcacca

SEQ ID NO: 299 - DNA, 17 bp, Homo sapiens
cctcggccgattcacca

SEQ ID NO: 300 - DNA, 18 bp, Homo sapiens
gctgttgtcatacttgct

SEQ ID NO: 301 - DNA, 17 bp, Homo sapiens
ggctagcttggtttaag

SEQ ID NO: 302 - DNA, 17 bp, Homo sapiens
ccacgtgatgtagactg

SEQ ID NO: 303 - DNA, 18 bp, Homo sapiens
tgagtgtaacaggagatt

SEQ ID NO: 304 - DNA, 17 bp, Homo sapiens
aagggggttccttgggc

SEQ ID NO: 305 - DNA, 18 bp, Homo sapiens
cccggcctcgtcctagcc

SEQ ID NO: 306 - DNA, 16 bp, Homo sapiens
cggcctcgtcctagcc

SEQ ID NO: 307 - DNA, 17 bp, Homo sapiens
ttcccgggaaccttctc

SEQ ID NO: 308 - DNA, 17 bp, Homo sapiens
cctcggccgagtaacac

SEQ ID NO: 309 - DNA, 18 bp, Homo sapiens
caccctcggccgattcaa

SEQ ID NO: 310 - DNA, 17 bp, Homo sapiens
ccgattcaccacccggc

SEQ ID NO: 311 - DNA, 17 bp, Homo sapiens
gccgattaaccaccggc

SEQ ID NO: 312 - DNA, 17 bp, Homo sapiens
ctgatgtcgctgcgccc

SEQ ID NO: 313 - DNA, 18 bp, Homo sapiens
acacaatctgctaatagc

SEQ ID NO: 314 - DNA, 22 bp, Homo sapiens
tgagtgtaacaggagattaaca

SEQ ID NO: 315 - DNA, 20 bp, Homo sapiens
agtgtaacaggagattaaca

SEQ ID NO: 316 - DNA, 17 bp, Homo sapiens
ggccaacggtgcggtgg

SEQ ID NO: 317 - DNA, 16 bp, Homo sapiens
gccaacggtgcggtgg

SEQ ID NO: 318 - DNA, 17 bp, Homo sapiens
atcagagggctccaggt

SEQ ID NO: 319 - DNA, 18 bp, Homo sapiens
cgatctgtgtttgtctga

SEQ ID NO: 320 - DNA, 21 bp, Homo sapiens
taggtctcaatcagtattctc

SEQ ID NO: 321 - DNA, 17 bp, Homo sapiens
gcctgggtgctggctgg

SEQ ID NO: 322 - DNA, 16 bp, Homo sapiens
cctgggtgctggctgg

SEQ ID NO: 323 - DNA, 17 bp, Homo sapiens
ggaactctgttggtcta

SEQ ID NO: 324 - DNA, 21 bp, Homo sapiens
aaaacccagcctcgtacctag

SEQ ID NO: 325 - DNA, 17 bp, Homo sapiens
cccagcctcgtacctag

SEQ ID NO: 326 - DNA, 21 bp, Homo sapiens
ggccgattaacacccgcctcg

SEQ ID NO: 327 - DNA, 17 bp, Homo sapiens
gattaacacccgcctcg

SEQ ID NO: 328 - DNA, 18 bp, Homo sapiens
cagcctctacctagcctt

SEQ ID NO: 329 - DNA, 17 bp, Homo sapiens
tgcacagcatggccatg

SEQ ID NO: 330 - DNA, 28 bp, Homo sapiens
tcggccgattaacaccggcctcgtacct

SEQ ID NO: 331 - DNA, 16 bp, Homo sapiens
caccggcctcgtacct

SEQ ID NO: 332 - DNA, 21 bp, Homo sapiens
ggcctgtttgcttgtggtgca

SEQ ID NO: 333 - DNA, 18 bp, Homo sapiens
ctgtttgcttgtggtgca

SEQ ID NO: 334 - DNA, 17 bp, Homo sapiens
gaccattgagggcagca

SEQ ID NO: 335 - DNA, 24 bp, Homo sapiens
cctcggccgattaaccaccggcct

SEQ ID NO: 336 - DNA, 17 bp, Homo sapiens
cgattaaccaccggcct

SEQ ID NO: 337 - DNA, 24 bp, Homo sapiens
ctcggccgattaaccaccggcctc

SEQ ID NO: 338 - DNA, 17 bp, Homo sapiens
gattaaccaccggcctc

SEQ ID NO: 339 - DNA, 22 bp, Homo sapiens
aaaacccagcctcgtacctagc

SEQ ID NO: 340 - DNA, 17 bp, Homo sapiens
ccagcctcgtacctagc

SEQ ID NO: 341 - DNA, 19 bp, Homo sapiens
tcgtacctagccgtatgtt

SEQ ID NO: 342 - DNA, 18 bp, Homo sapiens
cctcgtccgattaccacc

SEQ ID NO: 343 - DNA, 17 bp, Homo sapiens
gcctcgtccgatcaccc

SEQ ID NO: 344 - DNA, 17 bp, Homo sapiens
ttgaaccctaggcatgt

SEQ ID NO: 345 - DNA, 17 bp, Homo sapiens
tgagacagctcatcaca

SEQ ID NO: 346 - DNA, 17 bp, Homo sapiens
tctggactgatctaaca

SEQ ID NO: 347 - DNA, 17 bp, Homo sapiens
caagttcctataggagt

SEQ ID NO: 348 - DNA, 17 bp, Homo sapiens
tgccataaactgggtta

SEQ ID NO: 349 - DNA, 23 bp, Homo sapiens
ggctaggtacgaggctgggtgtg

SEQ ID NO: 350 - DNA, 17 bp, Homo sapiens
ggctaggtacgaggctg

SEQ ID NO: 351 - DNA, 17 bp, Homo sapiens
aaacctgcaatatgatg

SEQ ID NO: 352 - DNA, 20 bp, Homo sapiens
gcgtgatggcggggggctct

SEQ ID NO: 353 - DNA, 15 bp, Homo sapiens
gcgtgatggcggggg

SEQ ID NO: 354 - DNA, 17 bp, Homo sapiens
gcttacatccgtgatgt

SEQ ID NO: 355 - DNA, 18 bp, Homo sapiens
ttactctcatgtggccaa

SEQ ID NO: 356 - DNA, 18 bp, Homo sapiens
tctgatgaacagaagaag

SEQ ID NO: 357 - DNA, 17 bp, Homo sapiens
tgtgatgagctgtctca

SEQ ID NO: 358 - DNA, 17 bp, Homo sapiens
tgttagatcagtccaga

SEQ ID NO: 359 - DNA, 17 bp, Homo sapiens
actcctataggaacttg

SEQ ID NO: 360 - DNA, 17 bp, Homo sapiens
taacccagtttatggca

SEQ ID NO: 361 - DNA, 23 bp, Homo sapiens
cacacccagcctcgtacctagcc

SEQ ID NO: 362 - DNA, 17 bp, Homo sapiens
cagcctcgtacctagcc

SEQ ID NO: 363 - DNA, 17 bp, Homo sapiens
catcatattgcaggttt

SEQ ID NO: 364 - DNA, 20 bp, Homo sapiens
agagccccccgccatcacgc

SEQ ID NO: 365 - DNA, 15 bp, Homo sapiens
cccccgccatcacgc

SEQ ID NO: 366 - DNA, 17 bp, Homo sapiens
acatcacggatgtaagc

SEQ ID NO: 367 - DNA, 18 bp, Homo sapiens
ttggccacatgagagtaa

SEQ ID NO: 368 - DNA, 18 bp, Homo sapiens
cttcttctgttcatcaga

SEQ ID NO: 369 - DNA, 19 bp, Homo sapiens
gctgaacctgcgactggta

SEQ ID NO: 370 - DNA, 17 bp, Homo sapiens
gctgaacctgcgactgg

SEQ ID NO: 371 - DNA, 17 bp, Homo sapiens
taggtacgaggctgggt

SEQ ID NO: 372 - DNA, 19 bp, Homo sapiens
ggctagtacgaggctgggt

SEQ ID NO: 373 - DNA, 17 bp, Homo sapiens
ggctagtacgaggctgg

SEQ ID NO: 374 - DNA, 19 bp, Homo sapiens
ctaggtacgaggctgggtg

SEQ ID NO: 375 - DNA, 17 bp, Homo sapiens
ctaggtacgaggctggg

SEQ ID NO: 376 - DNA, 17 bp, Homo sapiens
gtacgaggctgggtgtt

SEQ ID NO: 377 - DNA, 17 bp, Homo sapiens
gaaactgttggcgtgat

SEQ ID NO: 378 - DNA, 20 bp, Homo sapiens
gagcagaaacgggagacctg

SEQ ID NO: 379 - DNA, 17 bp, Homo sapiens
gagcagaaacgggagac

SEQ ID NO: 380 - DNA, 23 bp, Homo sapiens
ggccttcgagcggggtgttgggg

SEQ ID NO: 381 - DNA, 15 bp, Homo sapiens
ggccttcgagcgggg

SEQ ID NO: 382 - DNA, 17 bp, Homo sapiens
agtttcttcaagatcac

SEQ ID NO: 383 - DNA, 17 bp, Homo sapiens
gaggaagtaatctgccc

SEQ ID NO: 384 - DNA, 19 bp, Homo sapiens
taccagtcgcaggttcagc

SEQ ID NO: 385 - DNA, 17 bp, Homo sapiens
ccagtcgcaggttcagc

SEQ ID NO: 386 - DNA, 17 bp, Homo sapiens
acccagcctcgtaccta

SEQ ID NO: 387 - DNA, 19 bp, Homo sapiens
acccagcctcgtactagcc

SEQ ID NO: 388 - DNA, 17 bp, Homo sapiens
ccagcctcgtactagcc

SEQ ID NO: 389 - DNA, 19 bp, Homo sapiens
cacccagcctcgtacctag

SEQ ID NO: 390 - DNA, 17 bp, Homo sapiens
cccagcctcgtacctag

SEQ ID NO: 391 - DNA, 17 bp, Homo sapiens
aacacccagcctcgtac

SEQ ID NO: 392 - DNA, 17 bp, Homo sapiens
atcacgccaacagtttc

SEQ ID NO: 393 - DNA, 20 bp, Homo sapiens
caggtctcccgtttctgctc

SEQ ID NO: 394 - DNA, 17 bp, Homo sapiens
gtctcccgtttctgctc

SEQ ID NO: 395 - DNA, 23 bp, Homo sapiens
ccccaacaccccgctcgaaggcc

SEQ ID NO: 396 - DNA, 15 bp, Homo sapiens
ccccgctcgaaggcc

SEQ ID NO: 397 - DNA, 17 bp, Homo sapiens
gtgatcttgaagaaact

SEQ ID NO: 398 - DNA, 17 bp, Homo sapiens
gggcagattacttcctc